(19) United States
(12) Patent Application Publication
(10) Pub. No.: US 2024/0034994 A1
(43) Pub. Date: Feb. 1, 2024

(54) CULTURED SPHERICAL BODY AND MANUFACTURING METHOD THEREFOR

(71) Applicants: KYOTO UNIVERSITY, Kyoto (JP); SATAKE MultiMix Corporation, Toda-shi, Saitama (JP)

(72) Inventors: Haruhisa INOUE, Kyoto (JP); Keiko IMAMURA, Kyoto (JP); Takayuki KONDO, Kyoto (JP); Ngoc Anh Suong DANG, Kyoto (JP); Yoshikazu KATO, Toda-shi, Saitama (JP)

(21) Appl. No.: 18/258,707

(22) PCT Filed: Dec. 24, 2021
(86) PCT No.: PCT/JP2021/048306

§ 371 Date: Jun. 21, 2023

Related U.S. Application Data
(60) Provisional application No. 63/223,655, filed on Jul. 20, 2021.

(30) Foreign Application Priority Data
Dec. 24, 2020 (JP) 2020-215560

(51) Int. Cl. C12N 5/0793

(52) U.S. Cl. CPC C12N 5/0619; C12N 2506/45; C12N 2501/385

(57) ABSTRACT

A cultured spherical body that is capable of easily performing quantitative analysis of a region containing brain neurons, useful as a supply source of neurons for transplantation, and can be used suitably for drug discovery screening for neurological diseases and compound evaluation systems is obtained. Provided are a cultured spherical body including a first cell layer containing neural stem cells and/or neural progenitor cells and a second cell layer containing brain neurons, wherein the first cell layer is present in the superficial portion of the cultured spherical body, and a method for producing the cultured spherical body.

### TECHNICAL FIELD

The present invention relates to a cultured spherical body and to a method for producing the cultured spherical body.

### BACKGROUND ART

Methods for inducing pluripotent stem cells to differentiate into various types of cells have been developed, and the next goal is to induce them to differentiate into tissues and organs. The tissues and organs into which pluripotent stem cells have been three-dimensionally induced to differentiate are also referred to as “organoids”. Organoids are not only used as research targets for differentiation and disease mechanisms, but are also extremely useful in drug discovery and development and cell transplantation therapy.

A method is known for inducing mouse and human embryonic stem cells (ES cells) to differentiate into three-dimensional cultures having a structure similar to the multilayer structure found at an intermediate stage in the cerebral cortex of pregnancy in humans. Such cultures are called brain organoids, and Patent Literature 1 and. Non-Patent Literature 1 have reported that cell agglomerates are rapidly formed from a certain number of pluripotent stem cells dispersed in a serum-free medium (serum-free embryonic body quick method; SFEBq method), followed by suspension culture, so that brain organoids encapsulating a cerebral cortex-like structure in which the neuroepithelium forms the ventricular zone with the cortical plate in the periphery thereof and the limbic body laminated in this order are obtained. Then, a detailed analysis of the production process revealed that the rapid formation of the cell agglomerates promotes self-organization toward a cerebral cortex-like structure.

Furthermore, a method is known in which the agglomerates of pluripotent stem cells are subjected to suspension culture in the presence of a Wnt signal inhibitor and a TGFβ signal inhibitor, followed by further suspension culture under high oxygen conditions (see, for example, Patent Literature 2 and Non-Patent Literature 2). It has been reported that brain organoids encapsulating a cerebral cortex-like structure in which the subventricular zone, the intermediate zone, the subplate, the cortical plate, and the marginal zone are laminated in the periphery of the ventricular zone in this order and further having the basal ganglia, the choroidal tissue, and the hippocampus are obtained by this method from human ES cells. Furthermore, it has also been reported that human induced pluripotent stem (iPS) cells also can be induced to differentiate into similar brain organoids (see, for example, Patent Literature 3).

Furthermore, a method of differentiating human iPS cells to neuroectoderm, then embedding them in a three-dimensional matrix, and culturing them by shaking in a rotating bioreactor is known (see, for example, Patent Literature 4 and Non-Patent Literature 3). The method has showed that human iPS cells can be induced to differentiate into brain organoids having outer radial glial cells and also having structural features such as a cortical inner fibrous layer, Which has not been identified by previously known methods. This method is widely used as a method for producing brain organoids that are closest to a living brain.

### CITATION LIST

### Patent Literature

Patent Document 1: JP 2016-5465 A
Patent Document 2: WO 2015/076388 A
Patent Document 3: WO 2016/063985 A
Patent Document 4: WO 2014/090993 A

### Non-Patent Literature

Non-Patent Document 1: Cell Stem Cell. 2008 Nov. 6; 3 (5): 519-32
Non-Patent Document 2: PNAS Dec. 10, 2013 110 (50) 20284-20289
Non-Patent Document 3: Nature volume 501, pages 373-379 (2013)

### SUMMARY OF INVENTION

### Problem to be Solved by the Invention

Brain organoids are expected not only to be used as research targets for differentiation and disease mechanisms but also as a drug discovery screening system for neurological diseases, as an evaluation system for candidate compounds, and as a resource for neurons for cell transplantation therapy. In particular, since brain neurons are therapeutic targets for many intractable neurological diseases, there is a great need for imaging or physically extracting regions containing brain neurons within a brain organoid.

However, the cerebral cortex-like structure generally only partially occupies a brain organoid obtained by methods of conventional art. Furthermore, the proportion in the cortical plate is very small, and its shape and size greatly vary for each organoid (Patent Literature 1 to 4 and Non-Patent Literature 1 to 3). Therefore, there has been a problem that it is unsuitable for image analysis which is often used in high-throughput screening. For such an analysis, it is necessary to set a region (brain neuron layer) containing the desired brain neurons for each brain organoid as a region of interest (ROI), but it is not easy to automatically set the ROI due to the circumstances described above.

Under such circumstances, there has been a great demand for brain organoids that can be easily used for quantitative analysis of regions containing brain neurons and can be suitably used for drug discovery screening for neurological diseases and an evaluation system for compounds.

### Means for Solving the Problems

As a result of diligent studies, the inventors have found that a brain organoid having a structure that is greatly different from those obtained by the methods of conventional art, that is, a cultured spherical body in which neuroepithelium is present on the superficial layer of the organoid, and differentiated cells generated from the neuroepithelium occupy the interior of the organoid can be obtained by a predetermined culture method, thereby accomplishing the present invention.

That is, the present invention includes the following aspects.

[1]A cultured spherical body including a first cell layer containing neuralstem cells andior neural progenitor cells; and a second cell layer containing brain neurons, in which the first cell layer is present in a superficial layer of the cultured spherical body.
[2] The cultured spherical body according to [1], in which the first cell layer has an apical-basal polarity, with the apical side of the first cell layer present on the surface layer side of the cultured spherical body.
[3] The cultured spherical body according to [1] in which the brain neurons contain at least one neuron selected from GABAergic neurons, dopaminergic neurons, and hippocampal neurons.
[4] The cultured spherical body according to any one of [1] to [3], in which the second cell layer contains at least one neuron selected from spinal cord motor neurons and neural crest cells.
[5] The cultured spherical body according to any one of [1] to [4], in which the proportion of the first cell layer in the superficial layer of the cultured spherical body is not less than 30%.
[6] The cultured spherical body according to any one of [1] to [5], in which the proportion of the first cell layer in the superficial layer of the cultured spherical body is not less than 50%.
[7] The cultured spherical body according to any one of [1] to [6], in which when the angle formed by the migration direction of each neural stem cell and the primary cilia is referred to as 0 in the plurality of neural stem cells contained in the cultured spherical body, the values of randomly differ among the plurality of neural stem cells.
[8] A method for producing a cultured spherical body from pluripotent stem cells, including the steps of
(a) placing pluripotent stein cells in a culture tank with a stirring blade;
(b) unsteadily operating the stirring blade to perform unsteady stirring culture of the pluripotent stem cells in the presence of a pluripotent stem cell maintenance medium; and
(c) unsteadily operating the stirring blade to perform unsteady stirring culture of the pluripotent stem cells in the presence of a neuronal differentiation medium.
   [9] The method according to [8], in which the unsteady operation step includes an up-and-down reciprocating motion, a left-and-right reciprocating motion, a rotational motion with a variable speed or a rotational reciprocating motion of the stirring blade, in the steps (b) and (C).
   [10] The method according to [8] or [9], in which the unsteady operation step includes an up-and-down reciprocating motion of the stirring blade in the steps (b) and (c).
   [11] The method according to any one of [8] to [10], in which the unsteady operation step varies the operation of the stirring blade at an unsteady cycle in the range of 0.01 Hz to 100 Hz in the steps (b) and (c).
   [12] The method according to any one of [8] to [11], in which the unsteady operation step in the step (c) is performed for at least 10 days.
   [13] The method according to any one of [8] to [12], in which the neuronal differentiation medium contains retinoic acid or its derivative in the step (c).
   [14] A method for producing a cultured spherical body from pluripotent stem cells, including the steps of
(i) placing pluripotent stem cells or undifferentiated cells derived from pluripotent stem cells in a culture tank with a stirring blade; and
(ii) unsteadily operating the stirring blade to perform unsteady stirring culture of the pluripotent stein cells or undifferentiated cells in the presence of a neuronal differentiation medium,
   [15] The method according to [14], in which the unsteady operation step includes an up-and-down reciprocating motion, a left-and-right reciprocating motion, a rotational motion with a variable speed, or a rotational reciprocating motion of the stirring blade, in the step (ii).
   [16] The method according to [14] or [15], in which the unsteady operation step includes an up-and-down reciprocating motion of the stirring blade in the step (ii),
   [17] The method according to any one of to [16], in which the unsteady operation step varies the operation of the stirring blade at an unsteady cycle in the range of 0.01 Hz to 100 Hz in the step (ii).
   [18] The method according to any one of [14] to [7], in which the unsteady operation step in the step (ii) is performed for at least 10 days.
   [19] The method according to any one of [14] to [18], in which the undifferentiated cells form cell clusters.
   [20] The method according to any one of [14] to [19], in which the neuronal differentiation medium contains retinoic acid or its derivative in the step (ii).
   [21]A cultured spherical body produced by the method according to any one of [8] to [20].
   [22] A pharmaceutical composition including the cultured spherical body according to any one of [1] to [7] and or a portion of the cultured spherical body.
   [23] A method for screening for a drug, including the steps of: (1) contacting the cultured spherical body according to any one of [1] to [7] and [21] with a test substance; (2) measuring a desired properly of the contacted cultured spherical body or its culture supernatant; and (3) comparing the measured property with the property of a cultured spherical body or its culture supernatant not contacted with the test substance.
   [24] The method according to [23], in which the desired property is selected from vial cell count, protein expression level, or RNA expression level in the cultured spherical body or its culture supernatant.
   [25] The method according to [23] or further including a step of preparing a section from the cultured spherical body, after the step of contacting the cultured spherical body with the test substance, in which the property of the section is compared in the measurement step and the comparison step.
   [26] A method for producing a spinal cord motor neuron, including the steps of:
(a) placing pluripotent stem cells in a culture tank with a stirring blade;
(b) unsteadily operating the stirring blade to perform unsteady stirring culture of the pluripotent stem cells in the presence of a pluripotent stem cell maintenance medium; and
(c) unsteadily operating the stirring blade to perform unsteady stirring culture of the pluripotent stem cells in the presence of a neuronal differentiation medium.
   [27] A method for producing a spinal cord motor neuron, lauding the steps of:
(i) placing pluripotent stem cells or undifferentiated cells derived from pluripotent stem cells in a culture tank with stirring blade; and unsteadily operating the stirring blade to perform unsteady stirring culture of the pluripotent stem cells or undifferentiated cells in the presence of a neuronal differentiation medium.
   [28] A method for producing a GABAergic neuron or its progenitor cell, including the steps of:
(a) placing pluripotent stein cells in a culture tank with a stirring blade;
(b) unsteadily operating the stirring blade to perform unsteady stirring culture of the pluripotent stem cells in the presence of a pluripotent stem cell maintenance medium; and
(c) unsteadily operating the stirring blade to perform unsteady stirring culture of the pluripotent stem cells in the presence of a neuronal differentiation medium.
   [29] A method for producing a GABAergic neuron or its progenitor cell, including the steps of
(i) placing pluripotent stein cells or undifferentiated cells derived from pluripotent stem cells in a culture tank with a stirring blade; and
(ii) unsteadily operating the stirring blade to perform unsteady stirring culture of the pluripotent stem cells or undifferentiated cells in the presence of a neuronal differentiation medium.

### Advantageous Effects of Invention

According to the present invention, a cultured spherical body (inverted brain organoid) in which a layer containing neural stem cells and; or neural progenitor cells is present in a superficial layer, and a layer containing brain neurons is present inside thereof can be obtained. In this inverted brain organoid, image analysis with the brain neuron layer as a region of interest (ROI) is extremely easily performed due to the structure described above and thus can be suitably used for screening a drug acting on brain neurons. Furthermore, since the layer containing brain neurons can contain neural cells in the periphery of the brain (such as spinal cord motor neurons and neural crest cells), it can also be anticipated as a cell supply source for cell transplantation therapy against various neurological diseases.

### BRIEF DESCRIPTION OF DRAWINGS

FIG. 1 is a view comparing an example of the production process according to the present invention with an example of a production process according to conventional art in chronological order on a method for producing a brain organoid from pluripotent stem cells. “A” represents common differentiation steps when producing a brain organoid from pluripotent stem cells. “B” represents differentiation and production steps according to the conventional art (Patent Literature 4), “C” represents differentiation and production steps according to Examples 1 and 2 of the present invention, and “D” represents differentiation and production steps according to Examples 3 to 6 of the present invention.

FIG. 2 is a vertical sectional view schematically showing air example of a culture tank that can be used in the method for producing a cultured spherical body of the present invention.

FIG. 3 is a horizontal sectional view taken along the line A-A in FIG. 2.

FIG. 4 shows the results of analysis of the positional relationships between the first cell layer and the second cell layer in brain organoids on Day 56 obtained by the method according to conventional art and the method of the present invention. A and B respectively represent typical images obtained by double immunohistochemical staining of equatorial sections of the brain organoids of Comparative Example 1 and Example 3 with a first cell layer marker (SOX2: red) and a second cell Diver marker (TUJ1: green). C and D are pattern diagrams showing SOX2-positive regions with diagonal lines by tracing SOX2-positive regions (first cell layer: red) and TUE-positive regions (second cell layer: green) respectively in A and B. As the reference signs in the figures, “1” represents SOX2-positive regions, and “2” represents TUE-positive regions. E shows the results of analysis of the percentage of the SOX2-positive regions in the area up to a depth of 100 μm from each organoid surface (Mean±SEM, ****p<0.0001, Student's two-tailed t-test, conventional type: n 3, inverted type: n 7) by double immunohistochemical staining of equatorial sections of the organoids of Comparative Example 1 and. Example with SOX2 and TUJ1, referring to the type in which the SOX2-positive regions are mostly present inside the organoid as “ordinary brain organoid” and the type in which the SOX2-positive regions are mostly present on the superficial layer of the organoid as “inverted brain organoid”.

FIG. 5 shows the results of triple immunostaining and DAPI nuclear staining of the serial sections of the brain organoid obtained on Day 56 in Example 3 with SOX2, MAP2, and CTIP2. Images showing only SOX2 (red) and MAP2 (green) signals are shown in the right column, and images showing only CTIP2 (gray) and DAPI (blue) signals are shown in the left column. The numbers in the left side of each image represent serial section numbers, and the bar represents 500 μm.

FIG. 6 shows images obtained by multiple immunostaining of equatorial sections of the brain organoids on Day 56 (A and B) or Day 50 (C and D). FIG. 6A and FIG. 6B respectively show representative images obtained by quadruple immunostaining of the ordinary brain organoid of Comparative Example 1 and the inverted brain organoid of Example 3 with SOX2 (blue), TBR2 (gray), CTIP2 (red), and MAP2 (green). The red dashed line in each image represents the perimeter of the brain organoid. FIG. 6C and FIG. 61) respectively show representative images obtained by triple immunostaining of the ordinary brain organoid of Comparative Example 1 and the inverted brain organoid of Example 3 with SOX2 (red), MAP2 (green), and ZO1 (gray). FIG. 6E and FIG. 6F respectively show representative images obtained by triple immunostaining of the ordinary brain organoid of Comparative Example 1 and the inverted brain organoid of Example 3 with SOX2 (pink color), MAP2 (green), and N-cadherin (gray).

FIGS. 7a A and B in FIG. 7a respectively show typical images of the brain organoids on Day 56 obtained by double immunohistochemical staining and DAPI nuclear staining of Comparative Example 1 and Example 3 with MAP2 and a representative neuronal or glial cell marker in the cerebral cortex. The marker used was VGIut1 (Vesicular Glutamate Transporter 1, a glutamatergic neuron).

FIG. 7b respectively shows typical images of the brain organoids on Day 56 obtained by double immunohistochemical staining and DAPI nuclear staining of Comparative Example 1 and Example 3 with MAP2 and a representative neuronal or glial cell marker in the cerebral cortex. The markers used were GABA (gamma-aminobutyric acid, a GABAergic neuron) (C and D). ChAT (Choline Acetyltransferase, a cholinergic neuron) (E and F), TH (Tyrosine hydroxylase, a dopaminergic neuron) (G and H), GFAP fibrillary acidic protein, an astrocyte) (I and J), Iba1 (a microglia) (K and L), and 04 (an oligodendrocyte) (M and N).

FIG. 8 shows the results of analysis of the neuronal activity of the brain organoid on Day 56 of Example 3 using a multi-electrode array (MEA) system. A represents the experimental timeline. B is a representative phase-contrast microscope image of cells (brain organoid-derived cells) cultured on MEA chips. C is a graph showing the spike frequency before treatment (Pre), after PTX treatment, APS and CNQA treatment, and after washing (Washout). D is a chart of Rasters of the array-wide spike detection rate (AWSDR, spikes/s).

FIG. 9 shows typical images obtained by multiple immunostaining and DAN staining of sections of the brain organoids on Day 90 of Comparative Example 1 and Example 3 using a synapse-specific antibody. (A and B) Images by double immunostaining with GABA (red) and Bassoon (green: inhibitory post-synaptic marker). Arrows indicate GABA-Bassoon-double positive (yellow) dots. (C and D) Images by double immunostaining with SYN1 (red: presynaptic marker) and PSD-95 (green: excitatory post-synaptic marker). Arrows indicate SYN1-PSD-95-double positive (yellow) dots.

FIG. 10a shows the results of analysis of primary cilia of neural stein cells by multiple immunostaining of the brain organoids on Day 56 of Comparative Example 1 and Example 3 using an antibody specific to the primary cilia. A and B are each a 3D image constructed using IMAMS software by 3D-immunohistochemical staining with primary cilium (Arl13b) and neural stem cell (SOX2) markers and detecting signals using a two-photon excitation microscope (Nikon AIR MP). C to F show images obtained by 2D-immunohistochemical staining with primary ciliary cilium (Art 13b) and its basal body (Pericentrin: Pcnt) markers using a confocal laser scanning microscope (Olympus FV1000). E and F respectively show enlarged images within the frames of C and D. Arrows (Direction of differentiation & migration) in E and F indicate the directions in which cells differentiated from neural stem cells migrate. A, C, and E show the results for ordinary brain organoids, and B, D, and F show the results for inverted brain organoids.

FIGS. 10b G and U in FIG. 10b respectively show the results of analysis of a ratio of cells having primary cilia (G) and the length of the primary cilia in SOX2-positive cells (H),

FIG. 10c I in FIG. 10c is a pattern diagram showing the angle (Cilia angle) formed by the direction of migration of a cell differentiated from a neural stem cell and the cilium in the neural stem cell (SOX2-positive cell). The ellipse indicates the SOX2-positive neural stem cell, Primary cilium indicates the primary cilium of the neural stem cell, and the circle on the ellipse that serves the base end of the cilium indicates the cilium basal body. J in FIG. 10c is a graph showing the Cilia angle and percentage as measured in any three regions in the image obtained from any three organoids in the analysis of A or Bin FIG. 10a, The dashed line indicates the results of an ordinary brain organoid (Comparative Example 1), and a solid line indicates the results of an in veiled brain organoid (Example 3), respectively.

FIG. 10d includes images obtained by immunohistochemical staining of primary cilia (Ar113b, red) and the base (Pcnt, yellow) in the SOX2-positive cells of the primary cilia of the brain organoids of Comparative Example 1 (orbital mixing, K) and Example 7 (vertical mixing, L) at a higher magnification as compared with FIG. 10a. The direction from the yellow point (Pcnt) to the red line (Ar113b) is an index indicating the direction of the cilia of neural progenitor cells (SOX2, turquoise). The bar represents 50 tart

FIG. 10e includes images obtained by immunohistochemical staining of the cilia of the brain organoids of Comparative Example 1 (orbital mixing, M, 0, and Q) and Example 7 (vertical mixing, P, and R). M and N show the results of SOX2 staining, and O and P show the results of Pcnt and Ar113b staining. Q and R show the measurement results of the direction of the cilia. The bar represents 20 μm,

FIG. 11 includes graphs showing the results of analysis of the sphericity by periodically collecting cell clusters from the start of induction to differentiate into neurons. A indicates the results of analysis for Comparative Example 1, and B indicates the results of analysis for Examples 1 to 6. The numerical value described in each bar indicates the sphericity.

FIG. 12 show immunostaining images obtained by analyzing the accumulation of A13 oligomers in an inverted brain organoid produced from iPS cells derived from a healthy, subject (A) or a patient with familial Alzheimer's disease (B) using the method of Example 3. In both A and B, images in the right and left panels are images of the same field of view, in which images in the left panel are quadruple-stained images with A3 oligomers ed), MAP2 een), SOX2 (gray), and DAPI (blue), and images in the right panel are images displaying only the signals of A13 oligomers (red). The bar represents 500 μm.

FIG. 13 shows that vertical mixing has different physical properties from those from orbital mixing. A shows the results of numerical simulation of computational fluid dynamics for comparison of rheology parameters during stirring by vertical mixing at mixing speeds of 45, 60, and 75 minis with the rheology parameters during stirring by orbital mixing. The average of each parameter is displayed in the thumbnail of each panel. The flow rate and the shear stress were higher in orbital mixing than in vertical mixing. It was found that the turbulent energy and the energy dissipation related to the state of stirring power were higher in vertical mixing than in orbital mixing. B shows the results of comparison of the movement of spheres (white circles) in orbital mixing and vertical mixing by numerical simulation of DPM (Discrete Phase Model), which is an unsteady, solid-liquid multiphase flow model. It turned out that spheres during orbital mixing were distributed along the walls of the culture dish, gradually migrated toward the center, and gathered together, whereas spheres during vertical mixing uniformly dispersed throughout the culture bottle C shows a comparison of three-dimensional flow rates for orbital mixing (blue line) and vertical mixing (red line). It turned out that the movement of spheres during orbital mixing had higher flow rates in the X and Y directions than the movement of spheres during vertical mixing, and the flow rate in the Z direction was almost zero. In addition, the movement of spheres during vertical mixing showed rates with constant frequencies not only in the Y-Y direction but also in the Z direction. It was found that the drag on the spheres during orbital mixing was greater than during vertical mixing,

FIG. 14 shows the results of single-cell RNA sequencing of the brain organoids obtained by the production methods of Comparative Example 1 (orbital mixing) and Example 7 (vertical mixing). A shows dimensional reductions and clusters (PCA-UMAP) in all cells of the brain organoids obtained in Example 7 and Comparative Example 1. Org1, Org2, and Org3 represent three organoids generated by orbital mixing, and Org4, Org5, and Org6 represent three organoids generated by vertical mixing, respectively. B shows the results of analysis of the main markers of neurons and glial cells. PAX6 and SOX2 are markers of neural progenitors; FOXG1, MAP2, and NCAM1 are markers of neurons. BCLI1B (or CTIP2), TBR1, and SOX5 are markers of deep cerebral cortex, CUX2 and SATB2 are markers of superficial cerebral cortex; GRIA2 and SNAP2S are markers of glutamatergic neurons; GAD2, DLX1, and DLX5 are markers of GABAergic neurons; AQP4 and GFAP are markers of astrocytes; MAGI and OLIG2 are markers of oligodendrocytes; and CX3CR1 is a microglia marker.

FIG. 15a shows the results of analysis of the Heatmap of single-cell RNA sequencing data, showing a heat map of brain lesion-related genes in all cells.

FIG. 15b shows the results of analysis of the Heat map of single-cell RNA sequencing data, showing a heat map of genes altered in SOX2-positive cells.

FIG. 16 includes images showing the presence of GABAergic neurons in the brain organoids obtained by the production methods of Comparative Example 1 (orbital mixing) and Example 7 (vertical mixing). A shows the results of immunostaining of progenitor cells of GABAergic neurons on the ventral side on Day 56 with a specific marker (NKX2.1, cyan), The bar represents 200 NKX2.1-positive cells can be found in the inverted brain organoid of Example 7. B includes images of immunostaining of brain organoids on Day showing the results of double immunostaining of GABAergic neurons with a specific marker (GABA, red) and a general neuron marker (MAP2, green). The bar represents 50 μm. The inverted brain organoid of Example 7 was shown to be enriched for GABA-positive cells.

FIG. 17 shows immunostaining images obtained by analyzing the accumulation of Aβ oligomers in an inverted brain organoid produced from iPS cells derived from a healthy subject (A) or a patient with familial Alzheimer's disease (B) using the method of Example 7. Images in the left panel are quadruple-stained images obtained in the same conditions as those in FIG. 12, except that SOX2 was changed to PROX1, and images in the right panel are images displaying only the signals of Aβ oligomers (red). The bar represents 50 tam.

FIG. 18 includes images obtained by analyzing the organoids obtained by the production method of Comparative Example 1 (orbital mixing) without using Matrigel by SOX2 and MAP2 immunostaining. The bar represents 500 μm,

FIG. 19 shows the results of analysis (PCA-MAP) of gene expression by single-cell sequencing. NIKX2.1-positive cells (A) and PROX1-positive cells (B) were preferentially detected in organoids prepared by vertical mixing.

FIG. 20 shows generation of GABAergic neurons (A) and glutamatergic neurons (B) in the organoids obtained by the production methods of Comparative Example 1 (orbital mixing) and Example 7 (vertical mixing). The bar represents 50 μm.

FIG. 21 includes photographs of organoids in which the stirring method was changed from orbital mixing to vertical mixing on Day 15 from the start of the differentiation, showing damaged organoids (A and B).

FIG. 22 shows the growth rates of the organoids produced in Comparative Example 1 (orbital mixing) and Example 7 (vertical mixing), showing the quantitative results of organoid area (A) and perimeter (B) evaluated on the images.

FIG. 23 shows the results of evaluation of the culture period of unsteady stirring culture (vertical mixing) that is indispensable for preparation of inverted brain organoids and is a graph showing the relationship between the switch timing of the stirring conditions from vertical mixing to orbital mixing and the percentage of the SOX2-positive regions in the area up to 100 sun from the organoid surface.

FIG. 24 includes images obtained by immunostaining of organoids produced by vertical mixing on Day 34 and shows generation of the spinal cord motor neurons. A shows a representative image obtained by triple immunostaining with GFP (green), MAP2 (white), and DAPI (blue), B and C show representative images obtained by immunostaining with GFP and MAP2, respectively, D shows a representative image obtained by quadruple immunostaining with Met′ (red), Oligi2 (white), GFP (green), and DAPI (blue), and E, F, and G show representative images obtained by immunostaining with ISIet1, GFP and Oligi2, respectively. The bar represents 100 μm.

FIG. 25 includes images obtained by immunostaining of organoids produced by vertical mixing on Day 34 and show generation of dopaminergic neurons. A shows a representative image obtained by triple immunostaining with TH (red), MAP2 (green), and DAPI (blue), and B and C show representative images obtained by immunostaining with TH and MAP2, respectively. The bar represents 100 μm.

FIG. 26 includes images obtained by immunostaining of organoids produced by vertical mixing on Day 34, in which retinoic acid was added to the medium from Day 0, and shows generation of GABAergic neurons. A shows a representative image obtained by triple immunostaining with GABA (red), MAP2 (green), and DAPI (blue), and B and C show representative images obtained by immunostaining with GABA and MAP2, respectively. The bar represents 100 μm,

FIG. 27 includes images obtained by immunostaining of organoids produced by vertical mixing on Day 34, in which retinoic acid was added to the medium from Day 0, and shows generation of hippocampal neurons. A shows a representative image obtained by triple immunostaining with PROX1 (red), MAP2 (white), and DAPI (blue), and B and C show representative images obtained by immunostaining with PROX1 and MAP2, respectively. The bar represents 100 μm.

### MODE FOR CARRYING OUT THE INVENTION

Hereinafter, the embodiments of the present invention will be described. However, the present invention is not limited by the embodiments described below

### 1. Method for Producing Cultured Spherical Body

According to the first embodiment, the present invention relates to a method for producing a cultured spherical body. The method for producing a cultured spherical body according to the first aspect of the first embodiment includes the following steps:

(i) placing pluripotent stem cells or a cell cluster composed of undifferentiated cells in a culture tank with a stirring blade; and

(ii) unsteadily operating the stirring blade to perform unsteady stirring culture of the pluripotent stem cells or a cell cluster composed of undifferentiated cells in the presence of a neuronal differentiation medium.

As used herein, the cultured spherical body refers to a spherical tissue obtained by culturing pluripotent stem cells. In the cultured spherical body, the cells are self-organized. The culture obtained by the method according to the present invention includes a cultured spherical body in which the positional relationship between the first cell layer and the second cell layer constituting the culture is inverted, as compared with the culture obtained by the method according to the conventional art. Such a cultured spherical body may be refereed to as “inverted brain organoid”. The definitions of the first cell layer and the second cell layer will be described later.

In this description, a culture encapsulating one or more cerebral cortex-like structures to be obtained by inducing pluripotent stem cells to differentiate may be hereinafter referred to as “ordinary brain organoid”. In this description, such a “cerebral cortex-like structure” means a “structure in which neural stem cells and/or neural progenitor cells (that is, neural progenitors) form a ventricular zone-like structure, and a cell layer containing cerebral cortical neurons is present outside the ventricular zone-like structure”. Therefore, the “cerebral cortex-like structure” is found in ordinary brain organoids but is not found in inverted brain organoids.

In the present invention, pluripotent stem cells refer to stem cells having pluripotency capable of differentiating into all cell types present in a living body and having also proliferation potency. Examples of the pluripotent stem cells include embryonic stem (ES) cells, germline stem cells (“GS cells”), embryonic germ cells (“EG cells”), and induced pluripotent stem (iPS) cells. Among them, the cells are preferably ES cells and iPS cells, particularly preferably iPS cells. Furthermore, the organism from which pluripotent stem cells are derived is not particularly limited but may be mammals, preferably humans, horses, cows, mice, and rats, and particularly preferably humans.

In the present invention, pluripotent stem cells having a disease-related gene can be used for producing a brain organoid that serves as a pathology model. Examples of the disease-related gene include mutant genes such as presenilin 1 and presenilin 2 when the purpose is to produce an Alzheimer's disease model. Pluripotent stem cells having such a disease-related gene can be produced by initializing somatic cells having the gene according to a conventional method. For the somatic cells having the gene, patient-derived somatic cells of each disease may be used, and healthy person-derived somatic cells into which the disease-related gene is introduced or healthy person-derived somatic cells in which the gene is modified may be used. Furthermore, each pathology model may be a brain organoid produced using the method according to the present invention from pluripotent stem cells produced from somatic cells derived from sporadic patients with each disease. The brain organoid that serves as a pathology model is preferably a brain organoid derived from mouse or human pluripotent stem cells.

Furthermore, “a cell cluster composed of undifferentiated cells” in the present invention refers to an aggregate consisting of cells that have not been oriented for differentiation. Here, to be “not directed to differentiate” means that differentiation is not committed and means to exhibit the property of pluripotent stem cells when they are dissociated and dispersed from the state of cell clusters to the state of single cells. For example, a cell cluster obtained by stationary or suspension culture of pluripotent steer cells in the presence of an embryoid formation medium for 3 days, preferably for 2 days, further preferably for 1 day, can be said to be a cell cluster composed of undifferentiated cells, as a guide. In this description, a cell cluster composed of undifferentiated cells may be referred to also as a “sphere”.

Next, a culture tank with a stirring blade that can be used in steps (i) and (ii) will be described, in the present invention, the culture tank with a stirring blade is a culture tank capable of three-dimensional unsteady stirring culture. In particular, it is a culture tank capable of unsteady operating of the stirring blade. Here, the term “unsteady” means not unidirectional drive (operation)/motion (movement) of the stirring blade, but drive/motion with a variable speed/reciprocating motion/reverse motion. The speed variation includes stopping and restarting the operation of the stirring blade. In the present invention, it is preferable that cells or a sphere be three-dimensionally suspended and dispersed in a fluid medium in, for example, a culture tank, and the cells or sphere can repeat migration/rotation/deformation within the flow action generated due to the unsteady drive/motion of the stirring blade within the fluid.

More specifically, it is preferably a culture tank that allows an up-and-down reciprocating motion, a left-and-right reciprocating motion, a rotational motion, or a rotational reciprocating motion of the stirring blade, preferably a culture tank that allows an operation change at a predetermined unsteady cycle. Details of the operation and movement conditions of the stirring blade will be described together with the description of step (ii).

The culture tank with the stirring blade is not specifically limited, as long as it is capable of three-dimensional unsteady stirring culture and is capable of achieving the operation/movement as described above. For example, a three-dimensional suspension culture stirring apparatus, HiD4×4, commercially available from SATAKE CHEMICAL EQUIPMENT MFG., LTD., can be used, but there is no limitation to this example. Hereinafter, the structure of the culture tank with a stirring blade capable of unsteady operation of the stirring blade will be described by way of non-restrictive examples.

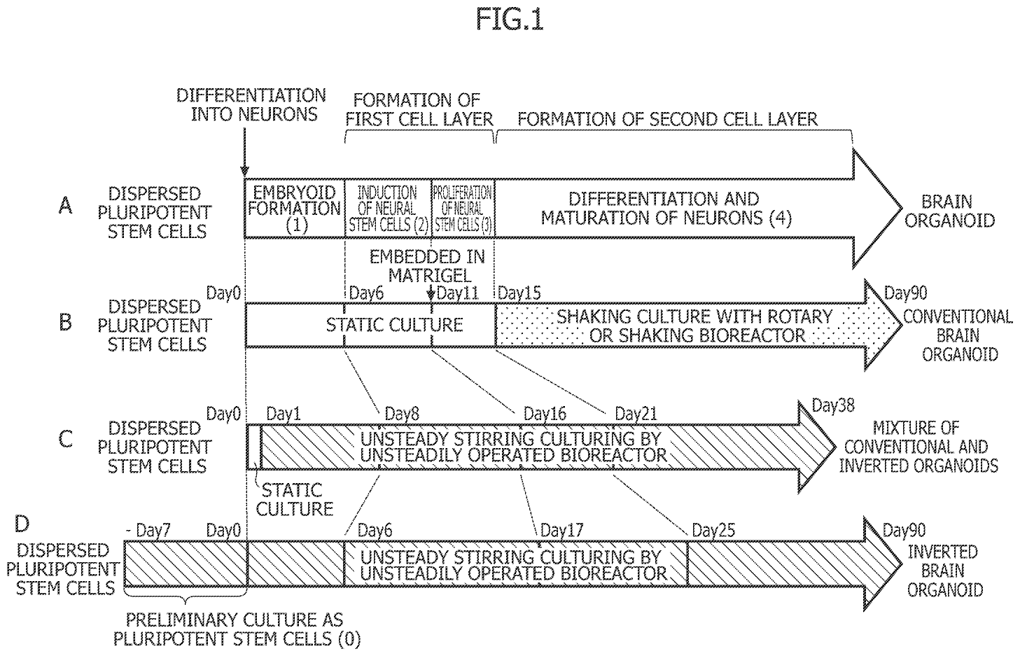

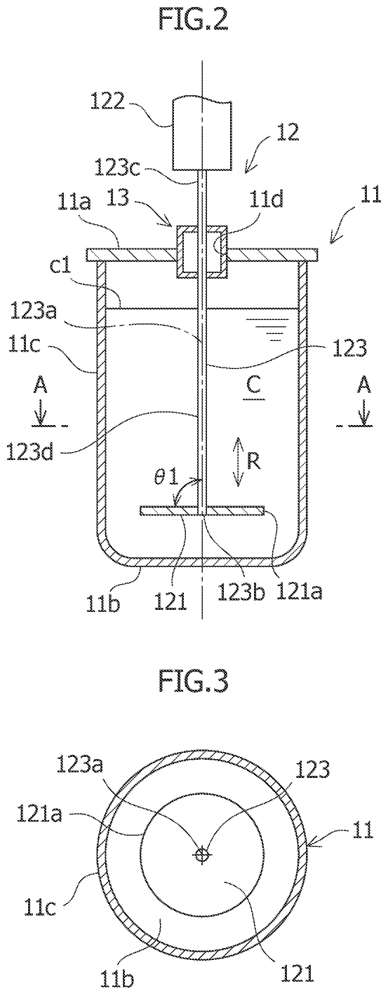

The culture tank shown in FIG. 2 and FIG. 3 includes a container 11 configured to contain a medium C, and a stirring mechanism 12 having one stirring blade 121 configured to stir the medium C in the container 11. The stirring mechanism 12 is configured to reciprocate the stirring blade 121. In FIG. 1, an arrow R indicates the reciprocating direction of the stirring blade 121, Furthermore, the stirring mechanism 12 controls the reciprocating motion of the stirring blade 121 so as to generate a desired shear stress in the medium C. In the stirring mechanism 12, the stroke of the reciprocating motion of the stirring blade 121, the speed of the reciprocating motion (such as the average speed of the reciprocating motion), the frequency of the reciprocating motion, and the like may be controlled. In particular, the reciprocating motion of the stirring blade 121 is preferably configured to be controllable in an unsteady pattern.

Furthermore, the production apparatus is preferably configured as follows. The container 11 of the production apparatus has a hollow body, and the container 11., in FIG. 2 and FIG. 3, as an example, is formed in a substantially cylindrical shape. In the present invention, the container may be formed in a shape other than the substantially cylindrical shape, as long as it has a hollow body. The container 11 has a top wall (or a top) 11a and a bottom wall (or a bottom) 11b, which face each other substantially vertically, and a peripheral wall (or a perimeter) 11c extending between the outer peripheral edges of the top wall 11a and bottom wall 11b. Furthermore, the container 11 is preferably formed in an elongated shape extending substantially vertically.

In FIG. 2, the top wall 11a is configured as a lid of the container 11 that is a separate body from a peripheral wall 11c, and the medium C can be put into the container 11 with the top wall 11a detached. In the present invention, an inlet for applying the medium may be formed in the container, and the top wall may be integrally formed with the peripheral wall in the container. In the present invention, depending on the production conditions of the brain organoid, the container may be formed so as to open upward, and an opening may be formed on the top wall, or the container may not have the top wall. The volume of the container 11 can be any value as long as it can produce a brain organoid but is preferably about not less than 50 mL, not less than 100 mL, not less than 200 mL, not less than 300 mL, not less than about 1 L, not less than about 50 L, not less than about 200 L, not less than about 500 L, not less than about 1000 L, or not less than about 2000 L, for example.

As shown in FIG. 2, the stirring blade 121 in the stirring mechanism 12 of the production apparatus is arranged along an intersecting plane that intersects the reciprocating direction at a predetermined intersection angle θ1. The intersection angle θ1 is about 90′, In other words, the stirring blade 121 is arranged along an intersecting plane that is substantially orthogonal to the reciprocating direction. The stirring blade 21 is formed in a substantially flat plate shape. An outer peripheral edge 121a of the stirring blade 121 is formed in a substantially circular shape, when viewed in a direction orthogonal to the intersecting plane. As shown in FIG. 2 and FIG. 3, the stirring blade 121 is arranged with a space from the top wall 11a, bottom wall lib, and peripheral wall 11c of the container 11. The stirring blade 121 may be referred to as an “impeller”. Furthermore, another shape of the stirring blade 121 and the distance between the peripheral wall 11c is of the container 11 and the outer peripheral edge 121a of the stirring blade 121 may be determined according to desired values of physical properties applied to cells or spheres.

However, the stirring blade of the present invention may have an intersection angle of the stirring blade of other than about 90°. The intersection angle may fall within the range of about 0° to about 180°. Furthermore, the stirring blade may be formed into a shape other than the substantially flat plate shape, depending on the desired physical property values applied to cells or spheres, or may be formed in a substantially hemispherical shell shape, a substantially bowl shape, a substantially curved plate shape, or a substantially corrugated plate shape, for example. Furthermore, the outer peripheral edge of the stirring blade may be formed into a shape other than the substantially circular shape, when viewed in a direction orthogonal to the intersecting plane, e.g., a substantially semicircular shape, a substantially elliptical shape, a substantially semi-elliptical shape, a substantially fan shape, and substantially polygonal shapes such as a substantially quadrangular shape, and a substantially star-shaped polygonal shape, when viewed in a direction orthogonal to the intersecting plane. Also, the stirring blade may have at least one hole penetrating in the reciprocating direction, and the shape, the number, and the position of the hole may be determined according to the desired physical property values.

As shown in FIG. 2, the stirring mechanism 12 has a driving source 122 for reciprocating the stirring blade 121 and a coupling member 123 that couples the stirring blade 121 to the driving source 122. The driving source 122 is configured to reciprocate the stirring blade 121 by reciprocating the coupling member 122. The driving source 122 may be configured to rotate the stirring blade 121 and the coupling member 123 about an axis 123a of the coupling member 123, in addition to the reciprocating motion. In this case, it is preferable that the stirring mechanism 12 control the rotational speed, the rotational direction, and the like of the stirring blade 121, and in particular, control the reciprocating motion and the rotation of the stirring blade 121 with an unsteady pattern, in addition to controlling the reciprocating motion of the stirring blade 121,

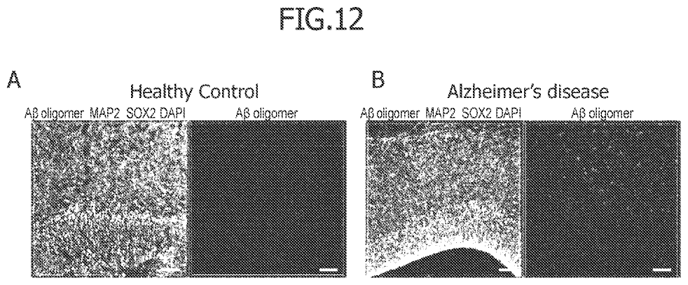

Furthermore, the coupling member 123 is formed into a substantially shaft shape extending along its axis 123a. A distal end 123b in the longitudinal direction of the coupling member 123 is attached to the stirring blade 121, and a proximal end 123c in the longitudinal direction of the coupling member 123 is held by the driving source 122 so as to be reciprocally movable. As shown in FIG. 12, the distal end 123b of the coupling member 123 is attached to a position substantially coinciding with the center of gravity of the stirring blade 121. The distal end of the coupling member may be attached to a position deviated from the center of gravity of the stirring blade,

The stirring mechanism 12 is attached to the top wall 11a of the container 11. As a specific mounting structure of the stirring mechanism 12, an insertion hole lid penetrating in the reciprocating direction is formed on the top wall 11a of the container 11, and the stirring mechanism 12 is attached to the top wall 11a of the container 11 with the stirring blade 121 contained inside the container 11 while the coupling member 123 is inserted through the insertion hole 11d. In the present invention, the stirring mechanism may be attached to the bottom wall or peripheral wall of the container instead of the top wall of the container by a specific mounting structure of the stirrer described above.

When trying to increase the airtightness of the container 11, the production apparatus may have a sealing member 13 configured to close the gap between the peripheral edge of the insertion hole 11d of the container 1 and the coupling member 123 of the stirring mechanism 12, while allowing the reciprocating motion of the coupling member 123. For example, the sealing member 13 may have a flexible structure that can follow the reciprocating motion of the coupling member 123. Furthermore, the flexible structure may have a membrane structure composed of a flexible material such as rubber, or the flexible structure may have a bellows structure composed of a metal. Teflon (Registered Trademark), and the like. In the present invention, the sealing member may hold the coupling member to be slidable in the reciprocating direction.

In the production apparatus, the stirring blade 121 of the stirring mechanism 12 reciprocates within a predetermined movable range in the container 11. The movable range is set in the container 11 or the medium C so that desired physical property values can be obtained. In particular, the length in the reciprocating direction of the movable range, that is, the maximum stroke of the reciprocating motion of the stirring blade 121 and the center position in the reciprocating direction of the movable range may be set depending on the length in the reciprocating direction of container 11, the distance from bottom wall 11b of the container 11 to a liquid surface cl of the medium C, the volume of the container 11, desired physical property values, and the like.

The culture tank is an example of the apparatus used in this embodiment, and the culture tank that can be used in this embodiment is not limited to those having a specific structure. It may be any bioreactor capable of three-dimensional unsteady stirring that can achieve fluidization and dispersion of cells while suppressing the shear stress that damages cells. Such unsteady stirring can also be referred to as vertical mixing (perpendicular mixing), and a preferable bioreactor can also be referred to as a vertical mixing-type bioreactor. Detailed conditions for operating the stirring blade will be described later.

Placing of pluripotent stem cells or spheres in the culture tank in step (i) can be carried out by a common method, Depending on the case, placing means adapted to the culture tank can be used. Furthermore, the concentration of placing can be set to an optimal placing concentration by one skilled in the art. In the present invention, pluripotent steal cells placed in the culture tank with a stirring blade in step (i) may be in the state of being dispersed into single cells, may be in the state of forming several to several tens of cell clusters (spheres), or may be in the state of a mixture thereof.

After placing the pluripotent stem cells or spheres, step (ii) is performed. Step (ii) is a step of unsteadily operating the stirring blade in the culture tank to perform unsteady stirring culture in the presence of a neuronal differentiation medium. In this embodiment, the neuronal differentiation medium means a medium that can be used for inducing pluripotent stem cells to differentiate into neurons, and the differentiation may be a step of inducing pluripotent stein cells to differentiate into neurons via embryoid bodies.

In the present invention, the neuronal differentiation medium may be a concept including a plurality of types of media. The differentiation from pluripotent stein cells into neurons can be preferably performed using mainly four types of media. These media can be used sequentially by replacement. Referring to FIG. 1A, an embryoid formation medium (1), a neural stem cell induction medium (2), a neural stem cell proliferation medium (3), and a neuron differentiation/maturation medium (4) can be used sequentially in chronological order of culture. The composition of the four types of media and the culture conditions for using the media will be described. Without using the neuron differentiation/maturation medium (4), the media (1), (2), and (3) may be sequentially used for culture, and even in this case, differentiation into various neurons can be induced. Furthermore, for specific neural cells (for example, spinal cord motor neurons and neural crest cells), only the media (1) and (2) may be used without using the media (3) and (4) as the neuronal differentiation media for production.

A basal culture medium can be used as the embryoid formation medium (1). The basal culture medium will be described later. The embryoid formation medium is preferably a basal culture medium to which serum and/or serum substitute is further added. The serum substitute can contain Knockout Serum Replacement, albumin, transferrin, fatty acid, collagen precursor, trace elements, 2-mercaptoethanol, 3″ thiol glycerol, or their equivalents, for example. Examples of a commercially available serum substitute include KSR (knockout serum replacement, Invitrogen) and Chemically-defined Lipid concentrated (Gibco), Furthermore, a basic fibroblast growth factor bFGF may be added thereto. Furthermore, an inhibitor (for example, Y-27632) of Rho-associated coiled-coil kinase (ROCK) is preferably added for the purpose of suppressing cell death of pluripotent stem cells induced by dispersion. Also, it is preferably substantially free from Wnt inhibitors and inhibitors.

Furthermore, it can contain other additives, without adversely affecting embryoid body formation and subsequent differentiation. Examples of the additives include insulin, iron sources (such as transferrin), minerals (such as sodium selenate), saccharides (such as glucose), organic acids (such as pyruvic acid and lactic acid), serum protein (such as albumin), amino acids (such as L-glutamine), reductants (such as 2-mercaptoethanol), vitamins (such as ascorbic acid and d-biotin), antibiotics (such as streptomycin, penicillin, and gentamicin), and buffers (such as HEPES), but there is no limitation to these examples.

Culturing in the embryoid formation medium (1) can be performed for about 4 to 10 days, preferably about 6 to 8 days. Culturing in the embryoid formation medium (1) can also be performed in the culture tank with a stirring blade from the start to the end. Alternatively, it is also possible to perform unsteady stirring culture after static culture in a culture dish or the like is performed for about 1 to 2 days from the start of culturing in the embryoid formation medium (1), and the resulting culture is transferred to the culture tank with the stirring blade.

As the neural stem cell induction medium (2), a basal culture medium can be used, preferably substantially free of serum or serum substitutes. The neural stem cell induction medium preferably contains an N2 supplement and may further contain heparin. Culturing in the neural stem cell induction medium (2) can be performed for about 6 to 15 days, preferably about 8 to 11 days. Culturing in the neural stem cell induction medium (2) is preferably performed in the culture tank with a stirring blade from the start to the end.

As the neural stein cell proliferation medium (3), a basal culture medium to which an N2 supplement and a B27 supplement (free from vitamin A) are added can be suitably used and may further contain insulin and Amphotericin B. The concentration of the N2 supplement in the neural stem cell proliferation medium is preferably lower than the concentration of the N2 supplement in the neural stem cell induction medium. Culturing in the neural stem cell proliferation medium (3) can be performed for about 4 to 10 days, preferably about 5 to 8 days. Culturing in the neural stem cell proliferation medium (3) is preferably performed in the culture tank with a stirring blade from the start to the end. After culturing using the neural stein cell proliferation medium (3) for about 4 to 10 days, culturing using the neural stem cell proliferation medium (3) can also be further performed without replacing with the neuron differentiation/maturation medium (4). Accordingly, when the medium (4) is not used, culturing using the neural stem cell proliferation median (3) may be performed for about 4 to 80 days.

As the neuron differentiation/maturation medium (4), a basal culture medium containing an N2 supplement, a B27 supplement (free from five types of antioxidants, vitamin E, vitamin E acetate, superoxide dismutase, catalase, and glutathione), insulin, and Amphotericin B is preferable. The neuron differentiation/maturation medium is preferably free from extracellular substrate proteins, particularly, Matrigel. Culturing in the neuron differentiation/maturation medium (4) can be performed for about 0 to 70 days, preferably about 5 to 65 days, further preferably about 10 to 50 days. Since the time course in which a cell is generated differs depending on the cell type, the culture period can be appropriately changed according to the purpose. Furthermore, the neural stem cell proliferation medium (3) can be used instead of the neuron differentiation/maturation medium (4). Culturing in the neuron differentiation/maturation medium (4) is preferably performed in the culture tank with a stirring blade from the start to the end. In some cases, culturing in the neuron differentiation/maturation medium (4) may not be performed.

Culturing sequentially using the media (1) to (3) is preferably performed for at least 14 days from the start of differentiation into a neuron, Day 0 to Day 14, more preferably at least 15 days from Day 0 to Day 15. When performing culturing for more than 14 days, the medium (3) can be used continuously, or it can be replaced with the medium (4).

The types and proportions of the cells contained in the second cell layer may van depending on the culture period. For example, a cell having pluripotency such as a neural crest cell is generated at an early stage, and the number of the cell remarkably increases from about Day 10 to Day 30. Therefore, for the purpose of producing neural crest cells, the culture period may be about 10 to 30 days. Furthermore, among neurons, a spinal cord motor neuron is generated early, and the number of the cell increases from about Day 14 to Day 40. Therefore, if the purpose is to produce a spinal cord motor neuron or its progenitor cell, the culture period may be about 14 to 40 days, preferably about 15 to 30 days. Since brain neurons tend to be generated later than spinal cord motor neurons, if the purpose is to produce a brain neuron, the culture period may be about 25 to 90 days, preferably about 30 to days, more preferably about 30 to 50 days. Since a glial cell tends to be generated later than a brain neuron, if the purpose is to produce a glial cell, the culture period may be about days or more, preferably 60 days or more. Among brain neurons, if the purpose is to produce a GABAergic neuron or its progenitor cell, or a hippocampal neuron or its precursor, the culture period may be about 30 to 90 days, preferably about 30 to 60 days. The culture period can be set to about 30 to 60 days by adding retinoic acid or its derivative, which will be described later, to the medium. Also, in culturing for the purpose of producing these cells, it is also possible to use the media (1) to (3) sequentially, and in culturing for more than 14 days, the media (1) to (4) can be sequentially used.

In the present invention, examples of the basal culture medium that can be used for differentiation from pluripotent stem cells into neurons include Glasgow's Minimal Essential Medium (GMEM), IMDM, Medium 199, Eagle's Minimum Essential Medium (EMEM), αMEM, Dulbecco's modified Eagle's Medium (DMEM), Ham's F12 medium, RPMI 1640 medium, Fischer's medium, Neurobasal Medium, DMEM/F12 medium, and mixed media thereof (for example, a mixed medium of Neurobasal Medium and DMEM/F12 medium), and the medium is not specifically limited as long as it can be used for culturing animal cells.

The basal culture medium may contain serum or may be free from serum. If necessary, the medium can contain one or more serum substitutes such as a serum substitute (such as Knockout Serum Replacement (KSR)), an N2 supplement (Invitrogen), a B27 supplement (Invitrogen), albumin, transferrin, apotransferrin, a fatty acid, insulin, a collagen precursor, a trace element, 2-mercaptoethanol, and 3′-thiol glycerol and may further contain one or more substances such as a lipid, an amino acid, L-glutamine, an alternative glutamine or L-glutamine dipeptide (for example, Glutamax (Invitrogen)), a non-essential amino acid (NEAA), a vitamin, a growth factor, a low-molecular weight compound, an antibiotic, an antioxidant, pyruvic acid, a buffer, inorganic salts, selenium acid, progesterone, and putrescine.

Among them, a preferable basal culture medium that can be used at any stage after induction of differentiation into neurons is DMEM/F12 medium. Furthermore, it is also possible to use a mixed medium with Neurobasal Medium at the differentiation/maturation stage (cerebral cortex formation stage) of neurons. The basal culture medium may contain serum and/or a serum substitute at the embryoid formation stage or may not contain these after the neural stem cell induction stage.

The production methods of Patent Literature 4 and Non-Patent Literature 3 require steps of coating cell clusters containing neural stem cells and/or neural progenitor cells with gel of a basement membrane matrix (Matrigel (Registered Trademark), Corning Inc.) and further culturing in a medium containing the basement membrane matrix. The method according to the present invention does not require such coating with the gel and there is no need to add Matrigel to the medium. The inventors have observed that, when culturing was performed without using Matrigel at all in the production methods of Patent Literature 4 and. Non-Patent Literature 3, a small amount of cultured spherical body with features of inverted brain organoids transiently occurred. However, it has also been confirmed that mature brain organoids cannot be obtained by these methods since the inverted cultured spherical body is very unstable and disintegrates within two months.

In this aspect, all media used (such as the media (1) to (3) or the media (1) to (4)) may preferably contain retinoic acid or retinoic acid derivatives in some cases. Culturing in a medium containing retinoic acid or its derivatives can remarkably increase the production rate of GABAergic neurons and hippocampal neurons, and increase the generated proportion of these neurons. The GABAergic neurons are neurons generated at a later stage in the occurrence of the cranial nervous system. For example, only about 30 days after the start of induction of differentiation into neurons, a culture containing GABAergic neurons and hippocampal neurons can be produced. In each of the media (1) to (4), the concentration of retinoic acid may be substantially the same as the concentration of retinoic acid commonly used in cell culture, and is preferably about 50 nM to about 1000 nM (unit), more preferably about 100 nM to about 1000 nM, and further preferably about 100 nM to about 300 nM, for example. When a retinoic acid derivative is used, a retinoic acid derivative having a concentration with the same activity as retinoic acid at the concentration can be used. Furthermore, the concentration of retinoic acid or derivatives of retinoic acid may be different or the same depending on the media (1) to (4). A retinoic acid derivative may be a derivative that has the differentiation function of retinoic acid. Examples of the retinoic acid derivative include 3-dehydroretinoic acid, 4-[[(5,6,7,8-tetrahydro-5,5,8,8-tetramethyl-2-naphthalenyl)carbonyl]amino]-benzoic acid (AM580) (Tamura K, et al., Cell Differ. Dev. 32: 17-26 (1990)), 4-[(1E)-2-(5,6,7,8-tetrahydro-5,5,8,8-tetramethyl-2-naphthalenyl)-1-propen-1-yl]-benzoic acid (TTNPB) (Strickland S, et al., Cancer Res. 41 5268-5272 (1983)), and the compound described in Tanenaga, K., et al., Cancer Res. 40: 914-919 (1980), retinol palmitate, retinol, retinal. 3-dehydroretinol, and 3-dehydroretinal, but this is not limited to these examples.

In this aspect, when the purpose is to produce a culture containing spinal cord motor neurons and neural crest cells, the medium preferably substantially free from retinoic acid or its derivative and further preferably substantially free from Hedgehog Signaling Activators (such as Smoothened Agonist). This is because when culturing is performed in a medium free from these, a culture containing numerous spinal cord motor neurons and neural crest cells can be obtained.

The culture temperature When performing step (ii) of the present invention is not specifically limited, but is about 30 to 40° C., preferably about 37° C. Culturing is preferably performed in an atmosphere of CO₂-containing air, and the CO₂ concentration is preferably about 2 to 5%. In the period for performing step (ii), the temperature and the CO₂ concentration are preferably constant, in culturing using any of the media (1) to (4).

In step (ii), unsteady stirring culture is performed in the culture period that is performed while sequentially changing the media, as described above. The unsteady stirring culture can be performed from the start to the end of the period for culturing using the media (1) to (3) or the media (1) to (4) continuously or intermittently. Preferably, it can be performed continuously.

The step of unsteadily operating the stirring blade includes a step of are up-and-down reciprocating motion, a step of a left-and-right reciprocating motion, a step of a rotational motion with a variable speed, or a step of a rotational reciprocating motion, or combinations of these of the stirring blade. Among them, the step of an up-and-down reciprocating motion of the stirring blade is particularly preferable. In step (ii) of the present invention, only one of these steps may be performed, or different operation steps may be combined during the culture period.

The step of an up-and-down reciprocating motion, the step of a left-and-right reciprocating motion, and the step of a rotational reciprocating motion of the stirring blade are all movements in which the stirring blade is moved in a finite orbit in the medium, which can be said to be movement including changes in the direction of movement. In general, the up-and-down movement can be referred to as movement in the vertical direction, and the-left-and-right movement can be referred to as movement in the horizontal direction.

The step of a rotational motion with a variable speed can be movement to rotate the stirring blade in an infinite orbit in the medium and vary the speed of rotation. The speed of rotation is preferably varied at a certain cycle, and this variation includes stopping operation and restarting operation. That is, even in the case in which the speed is zero, it is included in the variation.

The operation of the stirring blade refers to movement to vary the speed during vertical operation in an infinite orbit and movement to vary the speed during horizontal operation in an infinite orbit. Examples of the infinite orbit may be as described above.

In any of the movements described above, the unsteady operation can be at an unsteady cycle of 0.01 Hz to 100 Hz, and further preferably 0.5 Hz to 20 Hz. The unsteady cycle in the present invention is a cycle determined based on the maximum speed. That is, one cycle is from the point of giving the maximum speed to the point of giving the next maximum speed. The waveform may be sine waves, rectangular waves, triangular waves, waving waves, or the like, but there is no limitation to these examples. The waveform can be changed freely, and the cycle determined based on the maximum speed may be within such a range.

Using a vertical mixing-type bioreactor that can provide an environment to culture cells sensitive to a shear stress by such unsteady stirring, a homogeneous and stable environment can be maintained by continuously monitoring the culture conditions such as temperature, pH, and concentration of dissolved oxygen. This allows a so-called inverted brain organoid having an inverted structure in which neurons are localized in the center of the organoid, and neural progenitor cells cover the outside to be manufactured. That is, the production method according to the present invention suggests that differentiation of stem cells of a brain organoid is possible by controlling fluid mechanics by biomechanical engineering. Inverted brain organoids are anticipated to be applied to research on human brain development and disease, and are anticipated as a cell supply source that can be transplanted.

Unsteady stirring culture, that is, culturing by vertical mixing, may be performed for at least 10 days from the start of the nerve differentiation. Thereafter, inverted brain organoids can be produced even if it is switched to horizontal mixing (orbital mixing) with general horizontal rotation-type steady stirring (horizontal rotation on an orbital shaker). When the unsteady stirring culture period is shorter than 10 days, SOX2-positive cells may be significantly present the second cell layer, and the product may become an unfavorable culture as an “inverted brain organoid”. The unsteady stirring culture period is preferably 11 days, 12 days, 13 days, 14 days, or 15 days and may be more than that. Accordingly, the first aspect includes an unsteady stirring culture process for at least 10 days, preferably about 11 to 15 days, from the start of nerve differentiation, and unsteady stirring or steady stirring culturing is preferably continued until at least on day 14 from the start of nerve differentiation.

A method for producing a cultured spherical body according to the first aspect of the present invention will be described with reference to FIG. 1C. In the aspect shown in FIG. 1C, the cell cluster composed of undifferentiated cells obtained through static culture is placed in a culture tank with a stirring blade in step (i), In step (ii), the stirring blade is unsteadily operated, the cell cluster composed of undifferentiated cells obtained through static culture is cultured by unsteady stirring in the presence of a neuronal differentiation medium. It can be said that the production method according to FIG. 1C is an aspect in which single iPSC is cultured by unsteady stirring simultaneously with the start of nerve differentiation,

More specifically, a step of statically culturing pluripotent stem cells before step (i), at the start of the step of culturing using the embryoid formation medium (1), that is, from the time of replacement with the embryoid formation medium (1). The static culture step can be for 1 to 2 days. In general, this operation forms spheres. Then, the spheres obtained by the static culture step are placed in the culture tank with a stirring blade in step (i), and unsteady stirring culture is performed in step (ii). In step (ii), unsteady stirring culture is preferably performed by sequentially using media (1) to (3) or media (1) to (4). In the first aspect, the sphere subjected to unsteady stirring culture may be cell cluster composed of undifferentiated cells. The method for producing a cultured spherical body according to the first aspect shown in FIG. 1C allows a mixture of ordinary brain organoid and inverted brain organoid to be obtained.

Then, the production method according to the second aspect of the present invention includes the following steps of:
(a) placing pluripotent stem cells in a culture tank with a stirring blade;
(b) unsteadily operating the stirring blade to perform unsteady stirring culture of the pluripotent stem cells in the presence of a pluripotent stem cell maintenance medium; and
(c) unsteadily operating the stirring blade to perform unsteady stirring culture of the pluripotent stem cells in the presence of a neuronal differentiation medium.

In this aspect, after pluripotent stem cells are placed in the culture tank with a stirring blade, unsteady stirring culture is performed in the presence of a pluripotent stem cell maintenance medium before culturing using a neuronal differentiation medium. In this aspect, step (a) may be the same as step (i) of the first aspect, and step (c) may be the same as step (ii) of the first aspect. Accordingly, it is preferable to include an unsteady stirring culture process for at least 10 days from the start of nerve differentiation, preferably about 11 to 15 days and continue unsteady stirring or steady stirring culturing until at least on day 14 from the start of nerve differentiation also in the second aspect.

In step (b), the pluripotent stem cell maintenance medium is a medium used for the purpose of maintaining pluripotent stem cells and may be referred to as a preculture medium (0) in this embodiment. Preculture medium (0) may be a commercially available culture medium for pluripotent stem cells, preferably, iPS cells, and StemFit or the like to which a ROCK inhibitor is added can be used, for example. Step (h) described above of unsteady stirring culture pluripotent stem cells in the presence of a pluripotent stem cell maintenance medium can be referred to as a preculture step. Preculturing can be performed for about 1 to days, preferably about 2 to 8 days, and further preferably about 3 to 7 days. Furthermore, preculturing is preferably performed in the culture tank with a stirring blade from the start to the end. After the completion of preculturing, it is preferable to replace the culture medium with the embryoid formation medium (1), followed by unsteady stirring culture in the culture tank with a stirring blade.

With reference to FIG. 1D, the method for producing a cultured spherical body according to the second aspect includes a step (b) of preculturing pluripotent stem cells using preculture medium (0) in the culture tank with a stirring blade before step (c) of culturing pluripotent stem cells using the embryoid formation medium (1). That is, it includes step (b) of preculturing pluripotent stem cells before the start of the differentiation, After preculturing, the medium is replaced in the same stirring tank, and unsteady stirring culture sequentially using media (1) to (3) or media (1) to (4) in step (c) can be performed. Also in this aspect, it is preferable to add retinoic acid or its derivative to media (1) to (3) or media (1) to (4). Also in this aspect, for the purpose of obtaining a culture containing numerous spinal cord motor neurons and neural crest cells, the medium is preferably free from retinoic acid or its derivative, and preferably free of Smoothened Agonist when performing the method for producing a cultured spherical body according to this aspect. In this aspect, steps (b) and (c) can be carried out continuously in the culture tank without including the step of static culturing. According to the method for producing a cultured spherical body according to the second aspect, an inverted brain organoid can be selectively produced.

Then, the method for producing a cultured spherical body according to the first and second aspects of the present invention will be simply described in comparison with the method of production according to conventional art. FIG. 1B shows the method for producing a cultured spherical body disclosed in Patent Literature 4. In this method, static culturing using media (1), (2), and (3) described in the first aspect is performed, Matrigel is added to medium (4), and rotary or shaking culturing using this medium is performed with a bioreactor. Furthermore, when medium (2) is replaced with medium (3), the culture is embedded in the Matrigel. According to the production method of the conventional art, an ordinary brain organoid, that is, a culture in which a brain neuron layer is localized in the organoid superficial layer, and neural stem cell and neural progenitor cell layers are localized inside the organoid. In the production methods according to the first and second aspects of the present invention, it is preferable not to embed the culture in Matrigel. Furthermore, in the production methods according to the first and second aspects, any of media (1) to (4) is preferably free from Matrigel.

In the method for producing a brain organoid according to the conventional art particularly when performing the differentiation step in steady stirring culturing by orbital mixing, there has been a problem of a low amounts of brain organoid in the culture to be obtained. The brain organoid herein is a cultured spherical body at least including a neuroepithelium layer and a neuron layer. The culture obtained in conventional art generally contains unintended cultured material mainly composed of cells that do not belong to the neuronal lineage such as muscle tissue covering the skull, mesenchymal cells, and pigment epithelial cells. Therefore, in the case of using the culture for producing cells for transplantation, since a process to select only brain organoids from a wide variety of cultures (for example, a process of visually selecting brain organoids using tweezers under a microscope) is required, the work process becomes complicated, making it difficult to achieve automation, which is essential for mass production. In contrast, the method for producing a cultured spherical body according to the first and second aspects of this embodiment can provide a culture having a high proportion of brain organoids in the culture to be obtained and having a proportion of inverted brain organoids of at least 40%. This proportion can be 50% or more, 60% or more, 70% or more, 80% or more, 90% or more, 95% or more, or substantially all, depending on the case. Therefore, the resulting culture can be used as a supply source of neurons for transplantation as it is (without special process for selecting brain organoids) or after undergoing a selecting process based on automatable indicators such as size, shape, and specific gravity.

### 2. Cultured Spherical Body

The present invention relates to a cultured spherical body, according to the second embodiment. The cultured spherical body is produced by the production method according to the first embodiment and includes a first cell layer containing neural stein cells and/or neural progenitor cells and a second cell layer containing brain neurons, and the first cell layer is present in the superficial layer of the cultured spherical body. The cultured spherical body is preferably the culture obtained by the production method according to the second embodiment, after culturing in the presence of a neuronal differentiation medium for at least 14 days.

In the present invention, the term “spherical” means a structure with a sphericity of 0.8 or more, preferably 0.9 or more. The sphericity can be calculated using software based on an image of the cultured spherical body in a medium or in a liquid in which the shape of the cultured spherical body is not impaired. Specifically, it can be calculated by drawing the outline of the cultured spherical body using Image) software (polygon selection) and determining an area and a perimeter, according to formula: 4π*area/perimeter∧2.

The first cell layer contained in the cultured spherical body is a layer containing neural stem cells and/or neural progenitor cells. The neural stem cells and/or neural progenitor cells are also referred to as “neural progenitors”. In this description, secondary neural progenitor cells such as intermediate neural progenitor cells are not included in neural progenitors. In this description, the first cell layer containing “neural progenitors” may be referred to as “neural progenitor layer”, and the “neural progenitor layer” may contain cells other than neural progenitors. The neural progenitor layer is a cell layer corresponding to the neuroepithelium generated in mammalian neurogenesis and can be referred to also as a localized region of neural stem cells and/or neural progenitor cells. The first cell layer can be specified as a region densely populated with cells expressing marker genes for neural stem cells or neural progenitor cells, for example, by immunostaining, in situ hybridization, or the like. Examples of the marker genes for neural stem cells or neural progenitor cells include SOX2 and PAX6. Among these, SOX2 is particularly preferable.

The first cell layer has an apical-basal polarity, with the apical side facing the outside of the cultured spherical body (top surface). The fact that the apical side faces the outside c confirmed by analyzing the expression of N-cadherin, aPKC, ZO1, activated myosin (phosphorylated MLC2), and the like that are specifically expressed on the apical side of the neuroepithelium.

The second cell layer included in the cultured spherical body is a cell layer containing brain neurons. The term “brain neuron” means a “cell generated from a neural progenitor”. Accordingly, the second cell layer can also be referred to as a neuron layer generated from the neural progenitor layer. Specifically, examples of a cell contained in the brain neuron layer include constituent cells of the cerebral cortex such as the subventricular zone, the intermediate zone, the subplate, the cortical plate, and the marginal zone, and constituent cells of brain regions other than the cerebral cortex (basal ganglia, hippocampus, cerebellum, mesencephalon, optic nerve, and olfactory nerve). Among these, since the cerebral cortical neurons are prominently localized to the cortical plate, the second cell layer may include a cell layer corresponding to the cortical plate. The cell layer corresponding to the cortical plate does not have to form a layered structure like the living brain. Furthermore, the second layer includes the subventricular zone, the intermediate zone, the subplate, the cortical plate, and the marginal zone, in this order (relative order), from the side close to neuroepithelium toward the inside of the cultured spherical body in the case in which the subventricular zone, the intermediate zone, the subplate, the cortical plate, and the marginal zone are included in the second cell layer.

The second cell layer can be specified as a region in which cells expressing marker genes for general neurons or brain neurons are present, for example, by immunostaining, in situ hybridization, or the like. Examples of the marker genes for general neurons include TWA and MAP2, and TWA is particularly preferable. Examples of the marker genes for brain neurons include marker genes for neurons such as cerebral cortex, cerebellum, mesencephalon, midbrain, and basal ganglia, and examples of the marker genes for cerebral cortical neurons include FOXG1. The cerebral cortex may be further divided into a plurality of cell layers. For example, the cerebral cortex layer VI (Tbr1, Tbr2), layer V (Ctip2, Er81, Fezf2), layer IV (Rorb), and layer III/II (Foxp1, Mef2c, Satb2) in each of which characteristic neurons are localized may be included from the side close to the first cell layer toward the inside of the cultured spherical body. Marker genes often used for identification of each layer are shown in parentheses.

The second cell layer contains one or more types of brain neurons classified according to the main neurotransmitter type. Examples of the brain neurons include glutamatergic, cholinergic, dopaminergic, GABAergic, and serotoninergic neurons. Furthermore, the second cell layer also contains glial cells. Examples of the glial cells include astrocytes, oligodendrocytes, and microglia. The type and the composition of brain neurons and/or glial cells contained in the second cell layer can be controlled by changing the conditions of (ii) unsteady stirring culture. The second cell layer may further contain a spinal neuron and a glial cell. Examples of the spinal neurons include a motor neuron in the anterior horn of the spinal cord (lower motor neuron).

The culture according to this embodiment preferably contains al least one neuron selected from GABAergic neurons, dopaminergic neurons, and hippocampal neurons, among brain neurons. Therefore, it may contain only one of these, or any two or more types of cells. GABAergic neurons, dopaminergic neurons, and hippocampal neurons include the progenitor cells of GABAergic neurons, the progenitor cells of dopaminergic neurons, and the progenitor cells of hippocampal neurons. The presence of GABAergic neurons can be confirmed by immunostaining with GAD2, DLX1, and DLX5, the presence of the progenitor cells of GABA neurons can be confirmed by immunostaining with NKX2.1_, the presence of the progenitor cells of hippocampal neurons can be confirmed by immunostaining with PROX1, and the presence of dopaminergic neurons or their progenitor cells can be confirmed by immunostaining with TH, as markers.

The second cell layer preferably contains the neural cells of the surrounding tissue, which are cells other than brain neurons. The specific surrounding tissue may be, for example, the spinal cord. The neural cells of the surrounding tissue preferably contains at least one neuron selected from spinal cord motor neurons and neural crest cells, particularly spinal cord motor neurons. Accordingly, only one of these may be contained, or both of them may be contained. The presence of spinal cord motor neurons can be confirmed by immunostaining with HB9, ISL1 (Islet1) as markers, and the presence of neural crest cells can be confirmed by immunostaining with SOX10 as a marker, respectively.

In the cultured spherical body according to the present invention, that is, an inverted brain organoid, the proportion of the first cell layer in the superficial layer of the cultured spherical body is 30% or more, preferably 50% or more. Here, the superficial layer refers to a portion with a depth of 200 μm from the surface of the cultured spherical body, preferably up to 100 lam. The proportion of neural stem cells and; or neural progenitor cells refers to the area proportion showing SOX2 positive when preparing a section near the equator of the cultured spherical body, subjecting the section to 2D immunostaining, and observing it on an image.

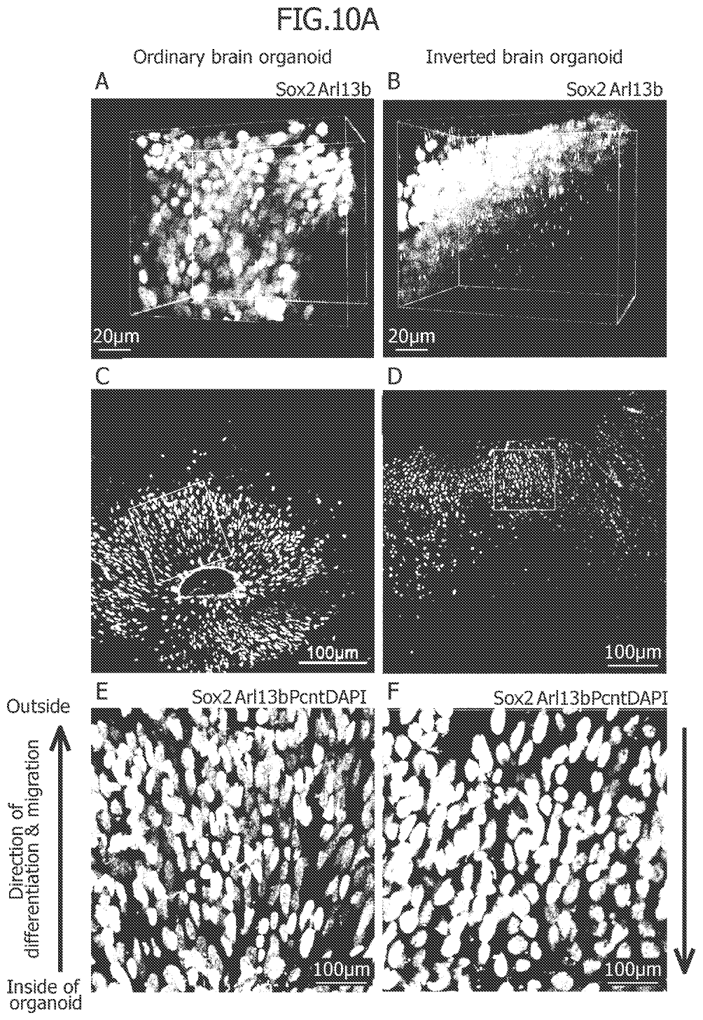

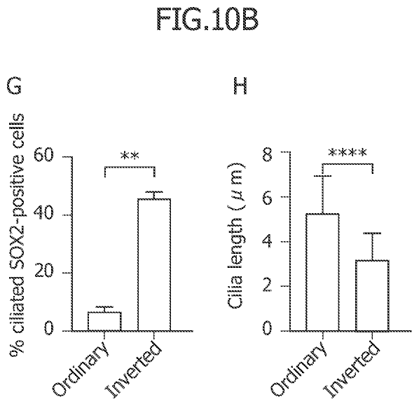

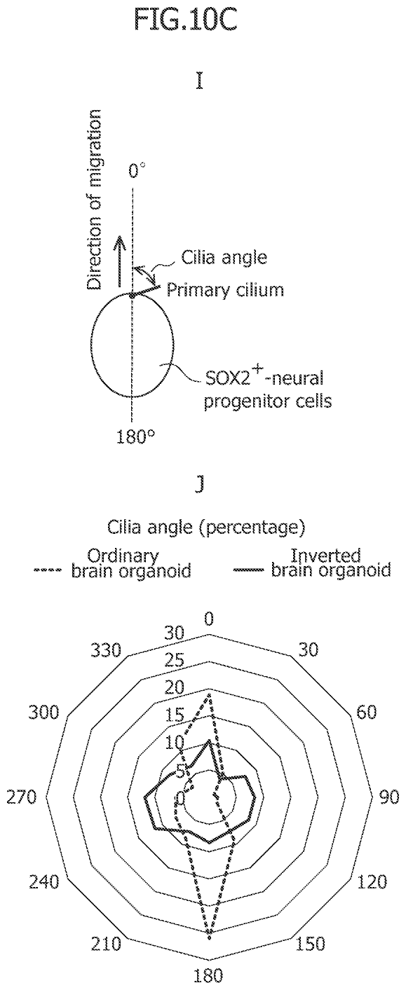

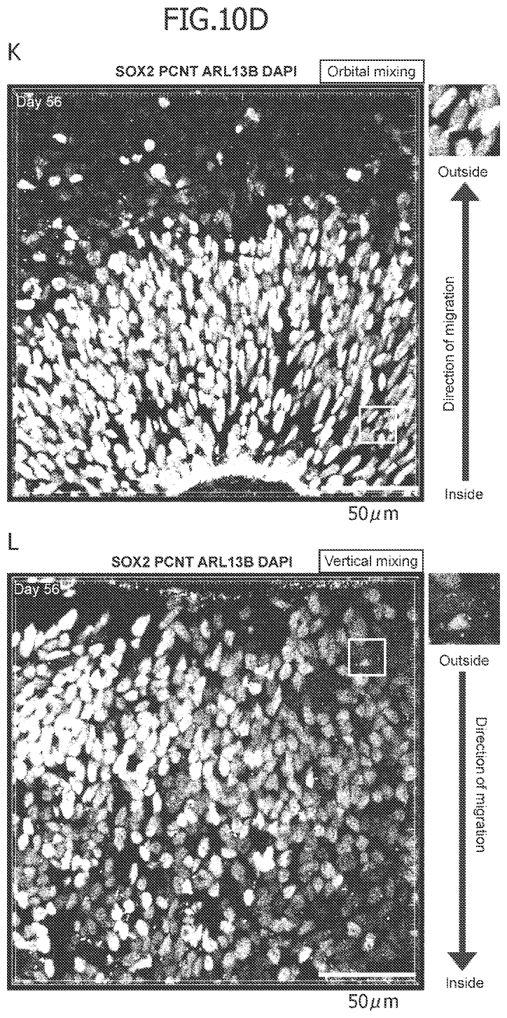

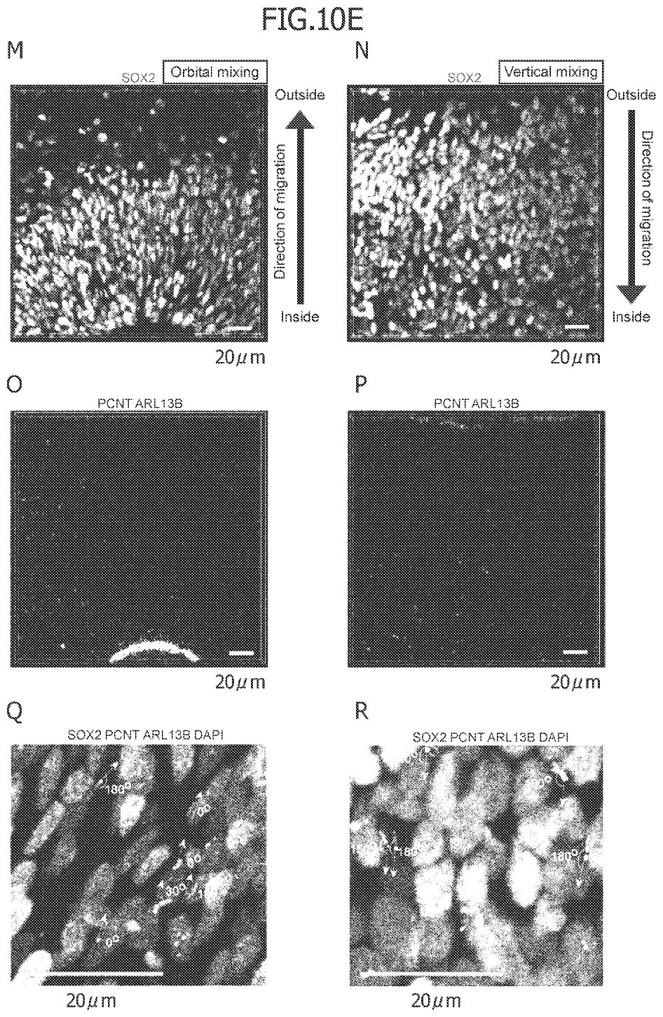

In a plurality of neural stem cells contained in the first cell layer of the cultured spherical body according to the present invention, when the angle formed by the migration direction of each neural stem cell and the primary cilia is referred to as 0, the values of 0 randomly differ between the plurality of neural stem cells. The angle θ formed by the migration direction and the primary cilia can be specified by measuring the angle of the primary cilia (Cilia angle shown by 1 in FIG. 10c) in any three regions on the image obtained from any three organoids. The fact that it differs randomly between the plurality of neural stein cells means that constituting the cultured spherical body, θ is distributed from 0″ to 360° substantially without deviation when θ is obtained for neural stem cells having the primary cilia. More specifically, when the frequency of the proportion of θ from 0″ to 360° is calculated for the plurality of neural stem cells, any one falls within 15%, and preferably 13%, of the total. The difference between the frequency of θ that is the maximum frequency and the frequency of θ that is the minimum frequency is within about 10%,

In the method for producing a brain organoid according to conventional art, similar to fetal cerebral cortex cells, the neuroepithelium composed of neural stem cells and/or neural progenitor cells (neural progenitors) has an apical-basal polarity and folds with the apical side facing inward to firm the ventricular zone (for example, Patent Literature 2 and Non-Patent Literature 2). Neurons originate from the outer surface (i.e., basal side) of the ventricular zone and migrate toward the surface of the culture. Therefore, the brain organoid produced by the method according to the conventional art has a cerebral cortex-like structure including a cerebral cortical the neuronal layer (the cortical plate in a narrow sense and the subventricular zone, the intermediate zone, the subplate, the cortical plate, and the marginal zone in a broad sense) generated outside the neural progenitor layer (ventricular zone) and basal ganglia and hippocampus and the like generated further outside thereof. Furthermore, the brain organoid produced by the method according to the conventional art is not necessarily spherical and often has a cerebral cortex-like structure as part of the brain organoid (Patent Literature 1 to 4 and Non-Patent Literature 1 to 3).

In contrast, the method according to the present invention can provide a cultured spherical body in which the neuroepithelium does not fold and is present in the superficial layer with the apical side facing outward. Then, neurons are generated from the basal side of the neuroepithelium and migrate toward the inside of the cultured spherical body, to generate a brain organoid that is a cultured spherical body in which the interior is substantially filled with neuroepithelium-derived cells. That is, a cultured spherical body in which the neural progenitor layer (neuroepithelium) is present in the superficial layer, and the brain neuron layer is further present inside thereof

### 3, Pharmaceutical Composition

The present invention relates to a pharmaceutical composition according to the third embodiment. The pharmaceutical composition includes a cultured spherical body produced by the production method according to the first embodiment of the present invention, a cultured spherical body according to the second embodiment, or a portion thereof. The whole culture can be used as it is as an active ingredient of the pharmaceutical composition.

A portion of the cultured spherical body can also be used as the active ingredient of the pharmaceutical composition. The portion may be, for example, a portion obtained by dividing the entire cultured spherical body into 2 to 8 portions. Alternatively, the portion may be a portion including substantially only the second cell layer of the cultured spherical body. A portion including only the second cell layer of the cultured spherical body can be prepared by subjecting the entire cultured spherical body further to unsteady stirring culture in the stirring tank with a stirring blade and scraping off the first cell layer.

The pharmaceutical composition may further contain other ingredients in addition to the cultured spherical body or a portion thereof, as required. Examples of such ingredients include excipients necessary for formulation according to the dosage form, storage stabilizing ingredients necessary for stable storage, and other medicinal ingredients. Examples of other medicinal ingredients include anti-inflammatory drugs, antibacterial agents, immunosuppressants, cell growth factors, and hormones.

### 4. Screening Method Using Cultured Spherical Body

According to the fourth embodiment, the present invention relates to a drug screening method, including the steps of

(1) contacting a cultured spherical body with a test substance;

(2) measuring a desired property of the cultured spherical body contacted or its culture supernatant; and

(3) comparing the property measured with the property of a cultured spherical body or its culture supernatant not contacted with the test substance.

In this embodiment, the cultured spherical body used for screening is the cultured spherical body produced by the production method according to the first embodiment or the cultured spherical body according to the second embodiment, which is a so-called inverted brain organoid. The cultured spherical body may be the entire body including the first cell layer and the second cell layer or may be a portion thereof. The definition of a portion may be as described for the active ingredients of the pharmaceutical composition above. The cultured spherical body may also be a healthy subject cell-derived cultured spherical body or a diseased subject cell-derived cultured spherical body, depending on the purpose of screening.

The test substance used in step (1) may be any substance that can function as a drug against the nervous system and can be appropriately set by one skilled in the art according to the purpose of screening. Examples of the test substance include agents for preventing or treating neurological diseases, survival-promoting agents for neurons, and compounds with low toxicity to the nervous system, but there is no limitation to these examples. The neurological disease may be, for example, a neurodegenerative disease, particularly, Alzheimer's disease. The step of contacting the test substance may include adding the test substance to a medium containing the cultured spherical body and culturing it for a predetermined period of time.

In step (2), the desired property of the cultured spherical body or its culture supernatant that has been contacted with the test substance are measured. The desired property is not particularly limited, but may be, for example, viable cell count, protein expression level, or RNA expression level in the cultured spherical body or its culture supernatant

The property of the cultured spherical body can be evaluated by analyzing the blocks of the entire cultured spherical bodies as they are or by cutting the blocks into several pieces. For example, the desired property can be analyzed by 3D immunostaining.

Alternatively, the property of the cultured spherical body can be evaluated by preparing a section of the cultured spherical body to evaluate the property obtained from the section. The section can be prepared from a desired site of the cultured spherical body such as a portion near the equator and the end portion. The thickness of the section can be appropriately determined in relation to the property to be evaluated and can be, for example, 5 to 30 μm.

Depending on the purpose, the property can be evaluated for the inside of the section, that is, a portion in which neurons are localized, or the surface portion of the section, that is, a portion in which neural stem cells or neural progenitor cells are localized. The inverted brain organoid according to the present invention has a structure in which neural stem cells or neural progenitor cells, or neurons are mostly localized in a cultured spherical body, as described earlier. Therefore, target cells are present at substantially the same relative position in a section at substantially the same point obtained from a cultured spherical body having substantially the same diameter or major axis. For example, in a section near the equator of a cultured spherical body, the basal membrane is present in the outermost layer, neural stem cells or neural progenitor cells are localized up to the superficial layer of the section, and neurons are localized inside the section. The superficial layer of the section refers to a region to a depth from the surface of up to 12% of the length of the diameter or major axis, when the length of the diameter or major axis of a section is taken as 100%, In an aspect, the superficial layer can refer to a region of a depth from the surface of 0 to 200 μm, preferably 0 to 100 μm. The inside of the section refers to a region deeper than the depth of the superficial layer, including the center of the section. One skilled in the art can appropriately set a region of interest through preliminary experiments or the like and make comparisons.

In step (3), the property is measured for a cultured spherical body not contacted with the test substance or its culture supernatant in the same manner and is compared with the property in (2). The diameter or major axis of the cultured spherical body to be compared is preferably substantially the same. Furthermore, when a section is used as a measurement target, a section in substantially the same port be selected as a section to be compared.

Such comparison enables, for example, a test substance with a significantly higher or lower viable cell count as a suitable drug to be extracted. Alternatively, a test substance with a low predetermined protein expression level or RNA expression level can be extracted as an appropriate drug.

According to one aspect of the drug screening method, drug screening used for prevention or treatment of Alzheimer's disease can be mentioned. In this aspect, a cultured spherical body derived from an Alzheimer patient is prepared and used as the cultured spherical body used in step (1). In step (1), examples of the aspect of contacting the cultured spherical body with the test substance include adding the test substance in a medium, followed by culturing for a desired number of days. In step (2), a section is prepared from the cultured spherical body derived from an Alzheimer patient contacted with the test substance. In the screening according to this aspect, agglomerates of peptides that have accumulated outside neurons and/or agglomerates of tau proteins that have accumulated mainly in neurons in the Alzheimer patient are evaluated. Therefore, it is preferable to prepare a section that allows analysis of the neuronal layer in this aspect. For evaluation, markers that specifically recognize Aβ peptides and markers that specifically recognize tau proteins can be used. In particular, strong signal clusters (Dot) obtained by immunostaining using these markers can be detected, and quantitative analysis can be performed based on the number of Dots. Then, candidates for prophylactic/therapeutic drugs can be selected from those having the number of Dots significantly reduced in the section treated with the test substance as compared with the section that has not treated with the test substance.

### 5. Method for Producing Spinal Cord Motor Neuron

According to the fifth embodiment, the present invention relates to a method for producing a spinal cord motor neuron. The method for producing a spinal cord motor neuron may substantially include the same steps as in the method for producing a cultured spherical body according to the second embodiment of the present invention. Accordingly, the production methods according to the first and second aspects are possible, as follows.

A method for producing a spinal cord motor neuron including the steps of:
(i) placing pluripotent stem cells or undifferentiated cells derived from pluripotent stem cells in a culture tank with a stirring blade; and
(ii) unsteadily operating the stirring blade to perform unsteady stirring culture of the pluripotent stem cells or undifferentiated cells in the presence of a neuronal differentiation medium, Second Aspect

A method for producing a spinal cord motor neuron including the steps of:
(a) placing pluripotent stem cells in a culture tank with a stirring blade;
(b) unsteadily operating the stirring blade to perform unsteady stirring culture of the pluripotent stem cells in the presence of a pluripotent stem cell maintenance medium; and
(c) unsteadily operating the stirring blade to perform unsteady stirring culture of the pluripotent stem cells in the presence of a neuronal differentiation medium,

Steps (i) and (ii) in the first aspect and steps (a), (b), and (c) in the second aspect are as described in detail in the second embodiment, and the descriptions thereof are omitted herein. In this embodiment, culturing the pluripotent stem cells or undifferentiated cells in the presence of a neuronal differentiation medium in step (ii) or (c) is performed for at least about 14 days, so that a culture containing a spinal cord motor neuron can be produced. The number of days of culture may be, for example, about 14 to 40 days or about 15 to 30 days.

In this culture period, it is also possible to culture the cells sequentially using neuronal differentiation media containing the embryoid formation medium (1), the neural stem cell induction medium (2), the neural stem cell proliferation medium (3), and the neuron differentiation/maturation medium (4). Depending on the number of culture days, it is also possible to produce a spinal cord motor neuron using only media (1) and (2) without using media (3) and (4), or using only media (1), (2), and (3) without using medium (4). As described above in the second embodiment, unsteady stirring culture may be performed for at least 10 days, and preferably 15 days, from the start of the differentiation during the culture period, and either unsteady stirring culture or steady stirring culturing may be performed during the remaining culture period. The spinal cord motor neuron can be confirmed with commonly used markers such as 11B9 and ISL1.

The step (ii) or (c) may be followed by a step of extracting a spinal cord motor neuron from the resulting culture and purifying U. The specific methods for extraction and purification may be any methods for extraction and purification of cells that are generally performed, without particular limitation. For example, a method of selecting a spinal cord motor neuron by a FACS (fluorescence-activated cell sorter) using antibodies against cell surface molecules can be used.

A spinal cord motor neuron that can be produced by this embodiment can be used for cell transplantation therapy for subjects suffering from amyotrophic lateral sclerosis (ALS).

### 6, Method for Producing GABAergic Neuron

According to the sixth embodiment, the present invention relates to a method for producing a GABAergic neuron or its progenitor cell. The method for producing a GABAergic neuron or its progenitor cell may include substantially the same steps as the method fir producing a cultured spherical body according to the second embodiment of the present invention. Accordingly, the production methods according to the first and second aspects are possible, as follows.

A method for producing a GABAergic neuron or its progenitor cell, including the steps of:
(i) placing pluripotent stem cells or undifferentiated cells derived from pluripotent stem cells in a culture tank with a stirring blade; and
(ii) unsteadily operating the stirring blade to perform unsteady stirring culture of the pluripotent stem cells or undifferentiated cells in the presence of a neuronal differentiation medium.

### Second Aspect

A method for producing a GABAergic neuron or its progenitor cell, including the steps of
(a) placing pluripotent stem cells in a culture tank with a stirring blade;
(b) unsteadily operating the stirring blade to perform unsteady stirring culture of the pluripotent stem cells in the presence of a pluripotent stem cell maintenance medium; and
(c) unsteadily operating the stirring blade to perform unsteady stirring culture of the pluripotent stem cells in the presence of a neuronal differentiation medium.

Steps (i) and (ii) in the First Aspect and steps (a), (b), and (c) in the Second Aspect are as described in detail in the second or fifth embodiment, and descriptions thereof are omitted herein. In this embodiment, the step of culturing the pluripotent stem cells or undifferentiated cells in the presence of a neuronal differentiation medium is performed for at least about 30 days, so that a culture containing a GABAergic neuron or its progenitor cell can be produced. The number of days of culture may be, tier example, about 25 to 90 days or about 30 to 60 days. Furthermore, when the nerves differentiation-inducing medium contains retinoic acid or its derivative, the number of days of culture may be about 25 to 60 days or about 25 to 50 days. The unsteady stirring culture of (ii) or (c) may be performed for at least 10 days, preferably 15 days, from the start of the differentiation, and steady stirring culturing may be performed during the subsequent culture period.

The step (ii) or (c) may be followed by a step of extracting a GABAergic neuron or its progenitor cell from the resulting culture and purifying it. Such a step may be performed by any methods for extraction and purification of cells that are generally performed without particular limitation, in the same manner as in the fifth embodiment.

A GABAergic neuron or its progenitor cell that can be produced by this embodiment can be used for cell transplantation therapy for subjects suffering from Alzheimer's disease.

### 7. Method for Producing Neural Crest Cell

According to the seventh embodiment, the present invention relates to a method for producing a neural crest cell. The method for producing a neural crest cell may include substantially the same steps as the method for producing a cultured spherical body according to the second embodiment of the present invention. Accordingly, the production methods according to the first and second aspects are possible, as follows.

### First Aspect

A method for producing a neural crest cell, including the steps of:
(i) placing pluripotent stem cells or undifferentiated cells derived from pluripotent stem cells in a culture tank with a stirring blade; and
(ii) unsteadily operating the stirring blade to perform unsteady stirring culture of the pluripotent stem cells or undifferentiated cells in the presence of a neuronal differentiation medium, and further including the step of culturing the pluripotent stem cells or undifferentiated cells in the presence of the neuronal differentiation medium for 10 days to 30 days.

### Second Aspect

A method for producing neural crest cells, including the steps of:
(a) placing pluripotent stem cells in a culture tank with a stirring blade;
(b) unsteadily operating the stirring blade to perform unsteady stirring culture of the pluripotent stem cells in the presence of a pluripotent stem cell maintenance medium; and
(c) unsteadily operating the stirring blade to perform unsteady stirring culture of the pluripotent stem cells in the presence of a neuronal differentiation medium, and further including the step of
culturing the pluripotent stem cells in the presence of the neuronal differentiation medium for 10 days to 30 days.

Steps (i) and (ii) in the first aspect and steps (a), (b), and (c) in the second aspect are as described in detail in the second or fifth embodiment, and descriptions thereof are omitted herein. In this embodiment, the step of culturing the pluripotent stem cells or undifferentiated cells in the presence of a neuronal differentiation medium is preferably performed for 10 days to 30 days. This is because the neural crest cells are transiently generated in the process of the differentiation from pluripotent stem cells. In this culture period, it is also possible to culture the cells sequentially using neuronal differentiation media containing the embryoid formation medium (1), the neural stem cell induction medium (2), the neural stem cell proliferation medium (3), and the neuron differentiation; maturation medium (4). Depending on the number of culture days, it is also possible to produce neural crest cells using only media (1) and (2) without using media (3) and (4), or using only media (1), (2), and (3) without using medium (4). The unsteady stirring culture of (ii) or (c) may be performed for at least 10 days, preferably 15 days, from the start of the differentiation, and steady stirring culturing may be performed during the subsequent culture period.

The step (ii) or (c) may be followed by a step of extractimz a neural crest cell from the resulting culture and purifying it, Such a step may be performed by any methods for extraction and purification of cells that are generally performed without particular limitation, in the same manner as in the fifth embodiment.

A neural crest cell that can be produced by this embodiment can be used for cell transplantation therapy for diseases in which peripheral neurons (such as sensory neurons and intestinal neurons) are damaged and for diseases in which cartilage and bone tissue are damaged. In particular, a mesenchymal stem cell obtained by inducing a neural crest cell to differentiate by a known method is being prepared for practical use for cell transplantation therapy for replenishing damaged cartilage, bones, and muscles, and the method according to the present invention is also beneficial as a supply source of a mesenchymal stem cell.

### EXAMPLES

The present invention is described further in detail by way of examples. The following examples are not intended to limit the present invention.

### I. Production of Brain Organoid

### Comparative Example 1

Along with the overview shown in FIG. 1B, brain organoid was produced.

(1) Embryoid Body Formation

Healthy subject-derived human iPS cells (201B7 strain) were statically cultured in an iPS cell maintenance medium (0) under feeder cell-free conditions to a confluence of 80% (generally, about 10 days after passage). The cells were collected from the culture dish by 0.5X TrypLE Select (Life Technologies Corporation, A12859-01) treatment (at 37° C. for 4 minutes) and suspended in iPS cell medium (0), to count the number of viable cells. The cells were collected as pellets by low-speed centrifugation treatment (at 1000 rpm for 3 minutes) and suspended in an appropriate amount of the embryoid formation medium (1)-No. 1 to obtain a cell suspension.

The cell suspension was placed in an ultra-low adhesion U-shaped 96-well plate at 9000 viable cells/150 μl/well. Thereafter, cell clusters were rapidly formed on the bottom of the wells by low-speed centrifugation treatment (1000 rpm for 3 minutes) and started to be incubated. (statically cultured′) (at 5% CO₂ and 37° C.). This day was designated as Day 0 (day when the induction of differentiation into neurons was started). On Day 3, half of the medium (75 μl) was replaced with the embryoid formation medium (1)-No. 2.

### (2) Induction of Neural Stem Cells

On Day 6, half of the medium (75 μl) was replaced with the neural stem cell induction medium (2), and thereafter, the medium was changed every other day.

### (3) Proliferation of Neural Stem Cells

On Day 11, cell clusters (cell clusters encapsulating the neuroepithelium) were embedded in Matrigel. Specifically, the cell clusters were mixed with chilled unpolymerized Matrigel and then incubated at 37° C. for 20 minutes to polymerize the Matrigel. The resulting Matrigel droplets (the Matrigel droplets encapsulating cell clusters) were transferred into the neural stem cell proliferation medium (3) and incubated (at 5% CO₂ and 37° C.). Thereafter, the medium was changed every other day.

### (4) Differentiation/Maturation of Neurons

On Day 15, the Matrigel droplets were transferred to a 6 cm culture dish. About 16 Matrigel droplets per 6 ml were adjusted to be in the neuron differentiation/maturation medium (4)-No. 1 per 6 cm culture dish, and shaking culturing was started at 60 rpm using a shaking bioreactor (Cell Shaker, CS-LR (Taitec, Product Code: 0081704-000) used together with large shaking rack WR-36361,R) set in an incubator (at 5% CO₂ and 37° C.). Thereafter, the medium was changed every 3 to 4 days. On Day 40, the medium was changed to the neuron differentiation/maturation medium (4)-No. 2, containing Matrigel, and thereafter the medium was changed every 3 to 4 days. Thereafter, on Day 70, the medium was changed to the neuron differentiation/maturation medium-No. 3, thereafter the medium was changed every 3 to 4 days, and shaking culturing was performed until Day 90 to obtain a brain organoid.

### Examples 1 and 2

Along with the overview shown in FIG. 1C, the brain organoids of Examples 1 and 2 were produced. A cell suspension in which the cells were suspended in the embryoid formation medium (1)-No. 1 was obtained from iPS cells (201B7 strain) by the same method as in the “(1) embryoid body formation” of Comparative Example 1, described above. The cell suspension was placed in a container for forming and culturing a spheroid (35 mm EZSPHERE dish, 4000-905SP, AGC TECHNO GLASS CO., LTD.) and was statically cultured for one day from Day 0 to Day 1, to form a sphere. On Day 1, the sphere was transferred to a bottle for stirring culturing, up-and-down unsteady stirring culture was started under the operational conditions described in Table 3. For up-and-down unsteady stirring culture, a HiD4×4 SATAKE VMOVE MIXER Single-use Bioreactor (SATAKE. CHEMICAL EQUIPMENT MFG., LTD.) was used, and a HiD4×4 dedicated bottle was used as the bottle for stirring culturing. Thereafter, the type of the medium was sequentially changed to the neural stem cell induction medium (2) on Day 8, to the neural stem cell proliferation medium (3) on Day 16, and to the neuron differentiation/maturation medium (4)-No. 1 on Day 21, and the up-and-down unsteady stirring culture was performed until Day 38, to obtain a brain organoid.

### Examples 3 to 7

Along with the overview shown in FIG. 1D, the brain organoids of Examples 3 to 7 were produced. Cell pellets were collected from iPS cells (201B7 strain) by the same method as in the “(1) embryoid body formation” of Comparative Example 1 described above. The pellets were suspended in iPS cell maintenance medium (0) and transferred into a bottle for stirring culturing, and the up-and-down unsteady stirring culture was performed for 7 days from Day −7 to Day 0 under the operational conditions described in Table 4.

Thereafter, the type of the medium was sequentially changed to the embryoid formation medium (1)-No. 1, on Day 0, to the neural stem cell induction medium (2) on Day 6, to the neural stem cell proliferation medium (3) on Day 17, and to the neuron differentiation/maturation medium (4)-No. 1, on Day 25, and the up-and-down unsteady stirring culture was performed until Day 90, to obtain brain organoids.

Table 1 shows the compositions of the media used in Examples and Comparative Examples and Table 2 shows the types of the media used in each step thereof. In Examples 1 to 7 of the present invention, cell clusters were not embedded in Matrigel, and a medium containing Matrigel was not used.

TABLE 1

| Medium | | Composition |
|---|---|---|
| (0) iPS cell maintenance | | StemFit AK02N supplemented with 10 μm Y-27632 |
| medium | | |
| (1) Embryoid | No. 1 | DMEM/F12 supplemented with 20% (v/v) Knockout Serum replacement, |
| formation medium | | 3% (v/v) FBS, 1% (v/v) Glutamax, 1% (v/v) NEAA, 1% P/S, 4 ng/ml |
| | | bFGF and 50 μM Y-27632 |
| | No. 2 | DMEM/F12 supplemented with 20% (v/v) Knockout Serum replacement, |
| | | 3% (v/v) FBS, 1% (v/v) Glutamax, 1% (v/v) NEAA, and 1% P/S |
| (2) Neural stem cell | | DMEM/F12 supplemented with 1% (v/v) N2 supplement, 1% (v/v) |
| induction medium | | GlutaMAX, 1% (v/v) MEM-NEAA, 1 μg/ml heparin and 1% P/S |
| (3) Neural stem cell | | a 1:1 mixture of DMEM/F12 and Neurobasal medium supplement with 0.5% |
| proliferation medium | | (v/v) N2 supplement, 1% (v/v) B-27 without vitamin A, 1% (v/v) |
| | | GlutaMAX, 0.5% (v/v) MEM-NEAA, insulin 2.5 μg/ml, 1% P/S and |
| | | 0.1% Amphotericin B |
| (4) Neuron | No. 1 | a 1:1 mixture of DMEM/F12 and Neurobasal medium supplement with 0.5% |
| differentiation/ | | (v/v) N2 supplement, 1% (v/v) B-27 without AO, 1% (v/v) |
| maturation medium | | GlutaMAX, 0.5% (v/v) MEM-NEAA, insulin 2.5 μg/ml, 1% P/S and |
| | | 0.1% Amphotericin B |
| | No. 2 | a 1:1 mixture of DMEM/F12 and Neurobasal medium supplement with 0.5% |
| | | (v/v) N2 supplement, 1% (v/v) B-27 without AO, 1% (v/v) |
| | | GlutaMAX, 0.5% (v/v) MEM-NEAA, insulin 2.5 μg/ml, 1% P/S, 0.1% |
| | | Amphotericin B and 1% Matrigel (growth factor reduced). |
| | No. 3 | a 1:1 mixture of DMEM/F12 and Neurobasal medium supplement with 0.5% |
| | | (v/v) N2 supplement, 2% (v/v) B-27 without AO, 1% (v/v) |
| | | GlutaMAX, 0.5% (v/v) MEM-NEAA, insulin 2.5 μg/ml, 1% P/S, 0.1% |
| | | Amphotericin B, 2% Matrigel (growth factor reduced) |

TABLE 2

| | | Comparative | | |
|---|---|---|---|---|
| Step | | Example 1 | Examples 1 and 2 | Examples 3 to 7 |
| (0) Preculturing | Period | | | About for 7 days |
| | Medium | | | Day(−7)-Day 7: |
| | | | | Medium (0) |
| (1) Embryoid | Period | About for 6 days | About for 8 days | About for 6 days |
| formation | Medium | Day 0-Day 4: | Day 0-Day 8: | Day 0-Day 6: |
| | | Medium (1)-No. 1 | Medium (1)-No. 1 | Medium (1)-No. 1 |
| | | Day 4-Day 6: | | |
| | | Medium (1)-No. 2 | | |
| (2) Neural stem cell | Period | About for 5 days | About for 8 days | About for 11 days |
| induction | Medium | Day 6-Day 11: | Day 8-Day 16: | Day 6-Day 17: |
| | | Medium (2) | Medium (2) | Medium (2) |
| (3) Neural stem cell | Period | About for 4 days | About for 5 days | About for 8 days |
| proliferation | Medium | Day 11-Day 15: | Day 16-Day 21: | Day 17-Day 25: |
| | | Medium (3) | Medium (3) | Medium (3) |
| | | * Start culturing by | | |
| | | embedding cell | | |
| | | clusters in Matrigel | | |
| (4) Neuron | Period | About for 75 days | About for 17 days | About for 65 days |
| differentiation/ | Medium | Day 15-day 40: | Day 21-Day 38: | Day 25-Day 90: |
| maturation | | Medium (4)-No. 1 | Medium (4)-No. 1 | Medium (4)-No. 1 |
| | | Day 40-day 75: | | |
| | | Medium (4)-No. 2 | | |
| | | Day 75-day 90: | | |
| | | Medium (4)-No. 3 | | |

| | | | |
|---|---|---|---|
| Total culture period after start of | About for 90 days | About for 38 days | About for 90 days |
| induction of differentiation | | | |

* In Comparative Example 1, Neuron differentiation/maturation (4) was performed by shaking culturing with a shaking bioreactor (60 rpm)

Tables 3 and 4 show the operational conditions for unstead stirring culture in Examples.

TABLE 3

| Test Example | | | Example 1 | Example 2 |
|---|---|---|---|---|
| Culture | Unsteady | State at the start of | Sphere | Sphere |
| conditions | stirring | stirring culture | | |
| | conditions | Starting time of stirring | Day 1 | Day 1 |
| | | culture | | |
| | | Density | 2.4 spheres/ml | 2.4 spheres/ml |
| | | volume of medium | 250 ml | 250 ml |
| | | Speed | 80 mm/sec | 100 mm/sec |
| | | Stroke | 20 mm | 20 mm |
| | | Frequency | 2 Hz | 2.5 Hz |

| | | | |
|---|---|---|---|
| | Culture period | For 38 days | For 38 days |
| Results | Type of brain organoid | Inverted and | Inverted and |
| | | conventional types | conventional types |
| | Diameter of brain organoid | About 1 to 2.5 mm | About 0.5 to 1 mm |
| | Proportion of inverted brain organoid | About 50% | About 50% |
| | in culture | | |

In the stirring conditions of Tables 3 and 4, a stirring blade with an elliptical shape was operated so as to reciprocate up and down mostly using the culture tank shown in FIGS. 2 and 3, Furthermore, the maximum movement speed during vertical reciprocation was 55 mints to 80 mm/s, and the stroke was set to 15 mm here, although the stroke may be variable according to the amount and level of the liquid.

TABLE 4

| Test Example | | | Example 3 | Example 4 | Example 5 | Example 6 | Example 7 |
|---|---|---|---|---|---|---|---|
| Culture | Unsteady | State at the start | Single cell | Single cell | Single cell | Single cell | Single cell |
| conditions | stirring | of stirring culture | | | | | |
| | conditions | Starting time of | −Day 7 | −Day 7 | −Day 7 | −Day 7 | −Day 7 |
| | | stirring culture | | | | | |
| | | Density | 6 × 10³ | 6 × 10³ | 1.5 × 10⁴ | 1.5 × 10⁴ | 1.0 × 10⁴ |
| | | | cells/ml | cells/ml | cells/ml | cells/ml | cells/ml |
| | | volume of | 250 ml | 250 ml | 250 ml | 250 ml | 250 ml |
| | | medium | | | | | |
| | | Speed | 60 mm/sec | 75 mm/sec | 60 mm/sec | 75 mm/sec | 60 mm/sec |
| | | Stroke | 15 mm | 15 mm | 15 mm | 15 mm | 15 mm |
| | | Frequency | 2 Hz | 2.5 Hz | 2 Hz | 2.5 Hz | 2 Hz |

| | | | | | | |
|---|---|---|---|---|---|---|
| | Culture period | For 97 days | For 97 days | For 97 days | For 97 days | For 97 days |
| Results | Type of brain | Inverted | Inverted | Inverted | Inverted | Inverted |
| | organoid | type | type | type | type | type |
| | Diameter of brain | About 2 to | About 2 to | About 2 to | About 2 to | About 2 to |
| | organoid | 3 mm | 3 mm | 3 mm | 3 mm | 3 mm |
| | Proportion of inverted | 90% or | 90% or | 90% or | 90% or | 90% or |
| | brain organoid in | more | more | more | more | more |
| | culture | | | | | |

### 2. Analysis of Brain Organoid

### (1) Determination of Conventional Type/Inverted Type

The cultures (brain organoids) obtained in Comparative Example 1 and Examples 1 to 7 were subjected to 2D-immunohistochemical staining by the method shown below to determine the conventional type/inverted type.

### Preparation of Brain Organoid Blocks

Brain organoids with a diameter of about not more than 1 mm were treated and fixed with 4% paraformaldehyde (at room temperature) for 15 minutes, and brain organoids with a diameter of about 1 to 3 mm were treated and fixed with 4% paraformaldehyde (at room temperature) for 30 minutes. The fixed brain organoids were washed with PBS, allowed to stand overnight in 30% sucrose, embedded in OCT compounds, and frozen. The blocks (brain organoids embedded in OCT compounds) obtained were stored at minus 80 degrees.

### 2D-immunohistochemical Staining

Cryosections with a thickness of 12 μm prepared from the brain organoid blocks were used. The cryosections were treated with a primary antibody (overnight at 4° C.) and a secondary antibody (2 hours at room temperature) according to a conventional method and then analyzed using a fluorescence microscope (confocal FV1000, Keyence BZ-9000 or IN Cell analyzer 6000). Table 5 below shows the primary antibody used and the dilution conditions. In the table, the column “Antibody” indicates the protein name that the antibody recognizes. As secondary antibodies, AlexaFlour 405, 488, 546, 594, and 647 conjugates (Invitrogen, 1:1000) were used.

TABLE 5

| Antibody | Code | Host | Dilution |
|---|---|---|---|
| SOX2 | MAB2018, | Mouse | 1:1000 |
| | R&D Sytem | | |
| Tuj1 | D71G9, Cell Signalling | Rabbit | 1:500 |
| | Technology | | |
| MAP2 | Ab5392, Abcam | Chicken | 1:3000 |
| TBR2 | Ab23345, Abcam | Rabbit | 1:1000 |
| CTIP2 | Ab18645, Abcam | Rat | 1:1000 |
| ZO1 | Ab59720, Abcam | Rabbit | 1:200 |
| GFAP | Z033401, Dako | Rabbit | 1:300 |
| ALDH1L1 | H00010840-M01, Abnova | Mouse | 1:300 |
| vGlut1 | 135 303, Synaptic Systems | Rabbit | 1:150 |
| GABA | A2052, Sigma | Rabbit | 1:300 |
| ChAT | AB144P, Chemicon | Goat | 1:100 |
| TH | AB152, Millipore | Rabbit | 1:100 |
| ARL13B | 17711-1-AP, Proteintech | Rabbit | 1:400 |
| Pericentrin | Ab28144, Abcam | Mouse | 1:200 |
| SOX2 | 14-9811-82, eBioscience | Rat | 1:400 |

### Results

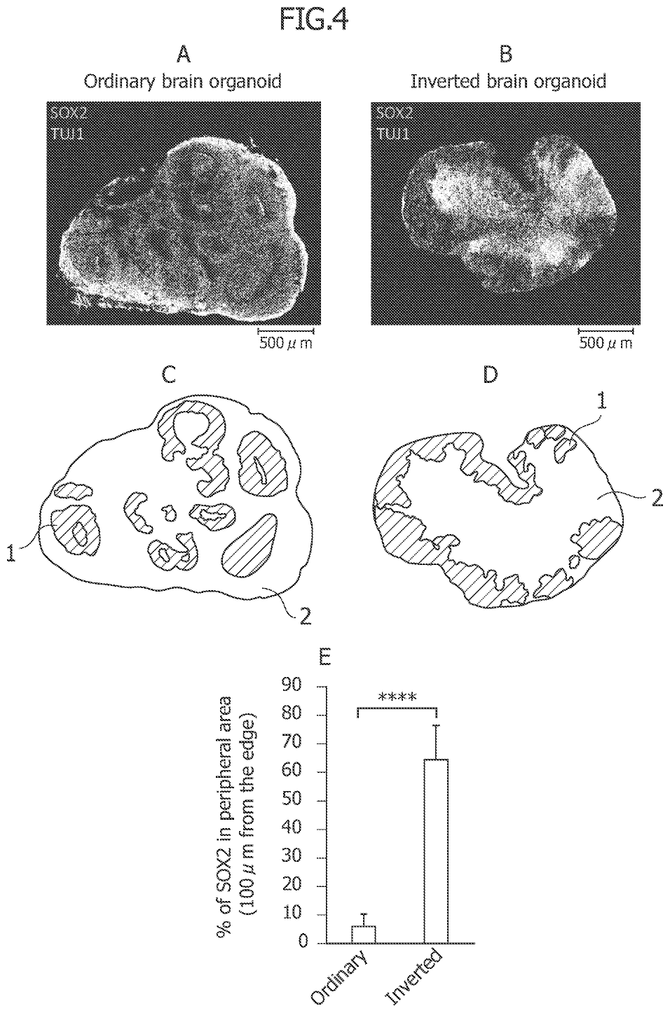

FIGS. 4A and 4B show the typical 2D-immunohistochemical staining images obtained for the brain organoids of Comparative Example 1 and Examples 3 to 6. In Wis. 4A and 4B, images tracing a SOX2-positive region (red: a cell layer containing neural stem cells and/or neural progenitor cells, that is, the first cell layer) and a TUE-positive region (green: a cell layer containing cerebral cortical neurons, that is, the second cell layer) are shown as FIGS. 4C and 4D.

In FIGS. 4A and 4C, it is understood that the first cell layer (red) forms a ventricular zone-like structure with a lumen, and the second cell layer green) is formed outside thereof, so that the first cell layer is basically present inside the brain organoid. In contrast, in FIGS. 4B and 4D, it can be found that the first cell layer (red) is present on the superficial layer of the brain organoid, and the second cell layer (green) is present inside thereof.

Referring to the organoid in which the first cell layer (red) is generally present inside the organoid, as shown in FIGS. 4A and 4C, to as a “conventional brain organoid”, and referring the organoid in which the first cell layer (red) is generally present on the superficial layer of the organoid, as shown in FIGS. 4B and 4D, to as an “inverted brain organoid”, the equatorial section of each organoid was analyzed for the proportion of the SOX2-positive region (red) in a region from the surface of the organoid to a depth of 100 μm. FIG. 4E shows the results.

As shown in FIG. 4E, the proportion of the SOX2-positive region (red) in the region from the surface of the organoid to a depth of 100 μm was only about 6% in the conventional brain organoid, whereas it was about 64% in the inverted brain organoid, which was significantly different. Therefore, it was clarified that the conventional type or the inverted type can be determined by the proportion of the first cell layer in the superficial layer of the organoid.

Table 3 and Table 4 also show the conditions of the unsteady stirring culture and the analysis results of the brain organoids obtained. In Examples 1 and 2, both the inverted type and the conventional type were observed, and in Examples 3 to 6, almost only the inverted type was observed.

Therefore, it was shown that inverted brain organoids can be produced by performing the unsteady stirring culture on the cell clusters composed of the pluripotent stem cells or undifferentiated cells in the presence of the neuronal differentiation medium using the culture tank with the stirring blade. Furthermore, it was clarified that the stirring blade is preferably moved reciprocally up and down in the unsteady stirring culture, and the operation of the stirring blade is preferably changed at an unsteady cycle of 0.01 Hz to 100 Hz, further preferably 1 Hz to 10 Hz.

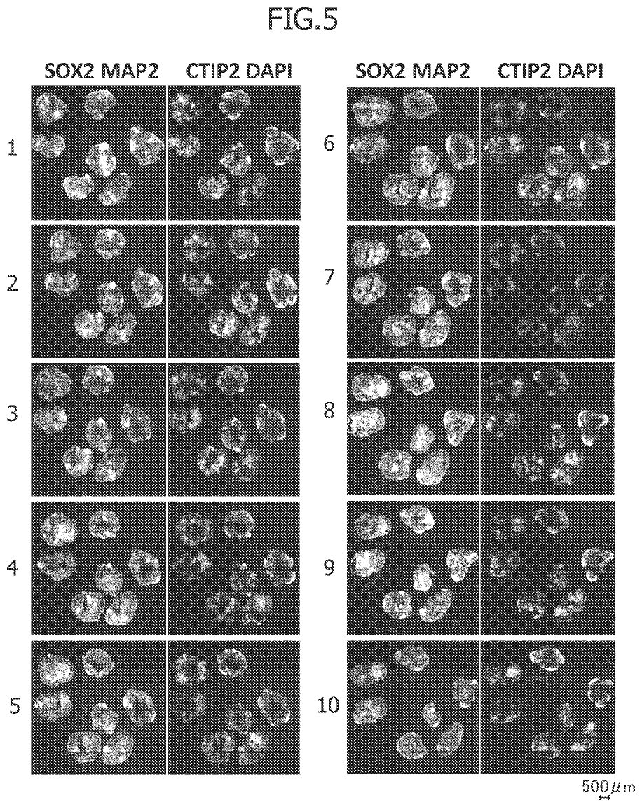

FIG. 5 shows the results of subjecting the serial section of the brain organoid obtained in Example 3, to triple immunostaining with SOX2, MAP2, and CTIP2, and DAPI nuclear staining. For easier viewing of the results, images displaying only the SOX (red) and MAP2 (green) signals were shown in the right column, and images displaying only the CTIP2 (gray) and DAPI (blue) signals are shown in the left column, separately. The number to the left of each image represents the number of the serial section, and the images in panels and 6 correspond to images near the equator.

In each brain organoid, it is understood that the SOX2-positive region (first cell layer) was present in the superficial layer, and the MAP2-positive region (second cell layer) was present inside the organoid, in the equatorial section (left images 5 and 6). Furthermore, it was also shown that part of the MPA2-positive region was a CTIP2-positive region (layer V of the cerebral cortex) (as compared with the left and right images).

Since the brain organoid is a very soft tissue, deformation during the fixation-embedding-sectioning stages is unavoidable, and therefore FIGS. 4A, 4B, and 5 show slightly distorted shapes. Then, since such deformation makes it difficult to determine the positional relationship between the first cell layer and the second cell layer in sections away from the equator, determinations of the conventional type/inverted type were performed using only the equatorial sections in this description.

### (2) Analysis of Each Cell Layer

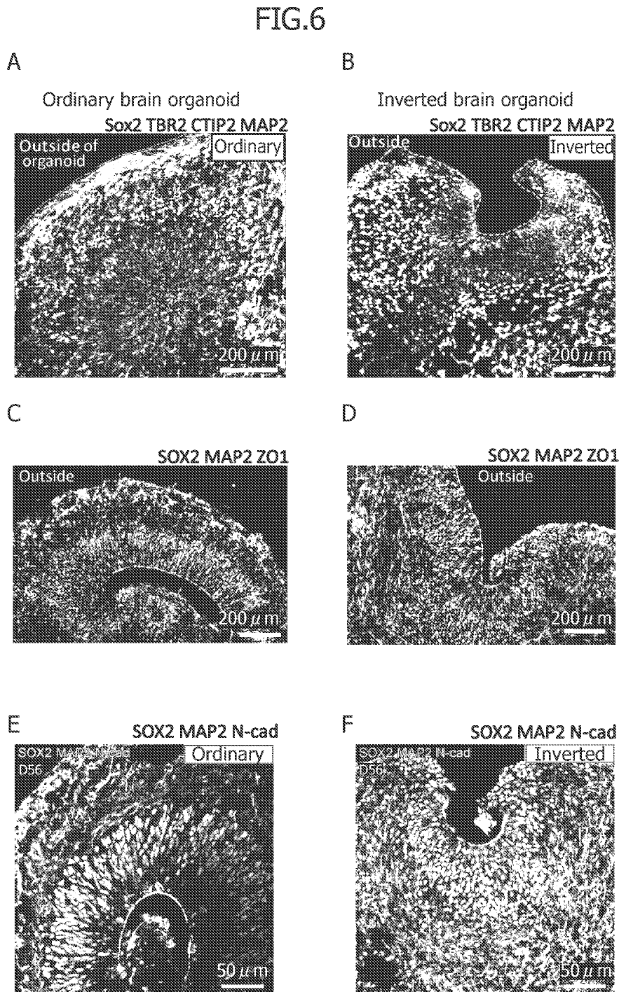

FIGS. 6A and 6B each show representative images obtained by 2D-quadruple immunohistochemical staining with SOX2 (blue), TBR2 (gray), CTIP2 (red), and MPA2 (green) of the brain organoids obtained in Comparative Example 1 and Examples.

As shown in FIG. 6A, TBR2-positive cells (gray: intermediate neural progenitor cells) were present in the perimeter of the SOX2-positive region (blue: first cell layer). CTIP2/MAP2-double positive cells (yellow to orange: neurons in layer V of the cerebral cortex) were present outside thereof, and MAP2-positive. CTIP2-negative cells (green: cerebral cortical neurons other than layer V) were present further outside thereof, in the conventional brain organoid. It was confirmed by this that the second cell layer included the subventricular zone (TBR2-positive), the deep cortical plate (CTIP2/MAP2-double positive), and the cortical plate other than the deep cortical plate (MAP2-positive CTIP2-negative), and they were formed outside the ventricular zone (first cell layer) in the same order as in the embryonic human brain.

In contrast, TBR2-positive cells (gray: intermediate neural progenitor cells) were present inside the SOX2-positive region (blue: first cell layer) in the organoid, CTIP2/MAP2-double positive cells (yellow to orange: neurons in the layer V of the cerebral cortex) were present inside thereof, and MAP2-positive CTIP2-negative cells (green: cerebral cortical neurons other than the layer V) were further present inside thereof in the inverted brain organoid, as shown in FIG. 6B. This strongly suggested that the subventricular zone (TBR2-positive), the deep cortical plate (CTIP2/MAP2-double positive), and the cortical plate other than the deep cortical plate (MAP2-positive CTIP2-negative) were formed also in the inverted brain organoid, but they occur from the first cell layer to the center of the organoid, in a manner opposite to an embryonic human brain.

Therefore, the polarity of the first cell layer was analyzed. FIGS. 6C and 6D show the results of triple immunostaining with SOX2 (red), MAP2 (green), and ZO1 (gray: apical marker of the neuroepithelium). In the conventional brain organoid (FIG. 6C), it was confirmed that the luminal side of the MAP2-positive region was ZO1.-positive, and the peripheral side of the first cell layer was the basal side. In contrast, the surface side of the organoid in the MAP2-positive region was ZO1-positive in the inverted brain organoid (FIG. 6D). It can be seen this that the organoid center side of the first cell layer was the basal side, and the superficial layer was the apical side in the inverted brain organoid.

Therefore, it was strongly suggested that the brain organoid produced by the method according to the present invention was of the inverted type in which the basal portion of the first cell layer faces the inside of the organoid, and thus, the second cell layer was formed inside the organoid.

### (3) Analysis of Neuronal and Glial Cell Types

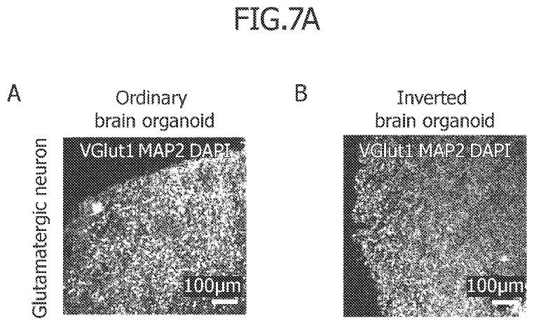

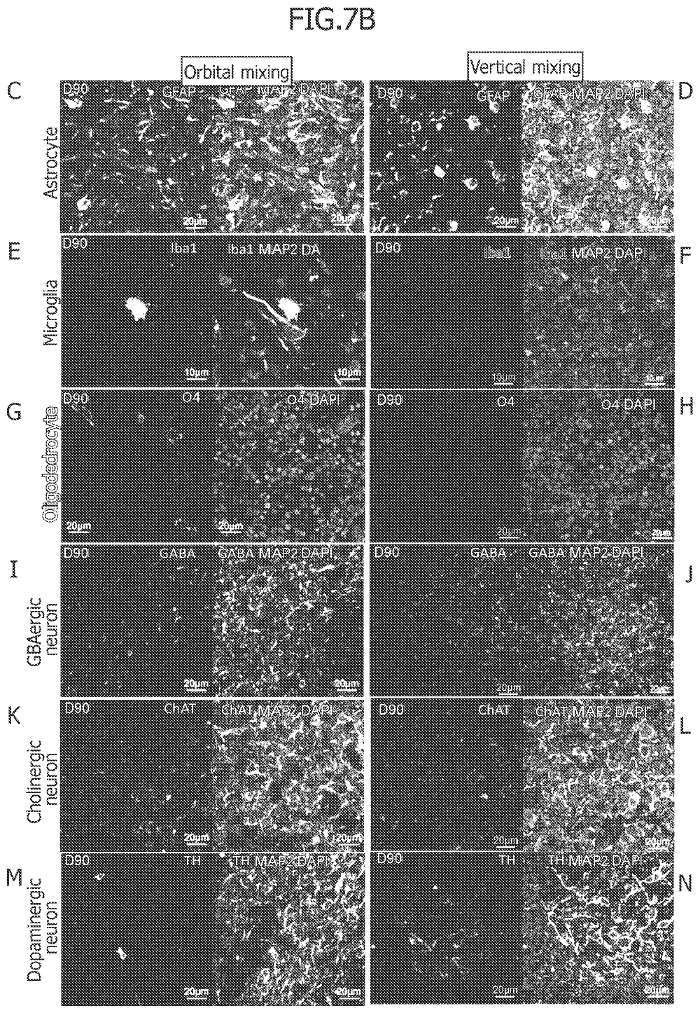

FIG. 7a shows the results of double immunohistochemical staining of the brain organoids obtained in Comparative Example 1, and Examples with MAP2 and cerebral representative neuronal or glial cell markers. As shown in A and B of FIG. 7a, a very large number of VGIut/MAP2-double positive cells (yellow to yellow green), that is, glutamatergic neurons were observed in the second cell layer in both the brain organoids. Furthermore, GABA/MAP2-double positive (yellow to yellow green) GABAergic neurons (C and D in FIG. 7b), ChAT/MAP2-double positive (yellow to yellow green) cholinergic neurons (E and F in FIG. 7b), TH/MAP2-double positive (yellow to yellow green) dopaminergic neurons (G and H in FIG. 7b) were also observed within the second cell layers of the conventional and inverted brain organoids. Among these, dopaminergic neurons are brain neurons that are not present in the cerebral cortex but in the midbrain. Furthermore, GFAP-positive MAP2-negative (red) astrocytes (I and J in FIG. 7b), Iba1-positive MAP2-negative (red) microglia (K and L FIG. 7b), and 04-positive MAP2-negative (red) oligodendrocytes (M and Nin FIG. 7b) were also observed within the second cell layer of the conventional and inverted brain organoids.

Therefore, it was confirmed that, also in the inverted brain organoid, various cerebral cortical neurons and glial cells occurred in the second cell layer, and brain neurons in the regions other than the cerebral cortex occurred, in the same manner as in the conventional brain organoid.

### (4) Analysis of Neural Function

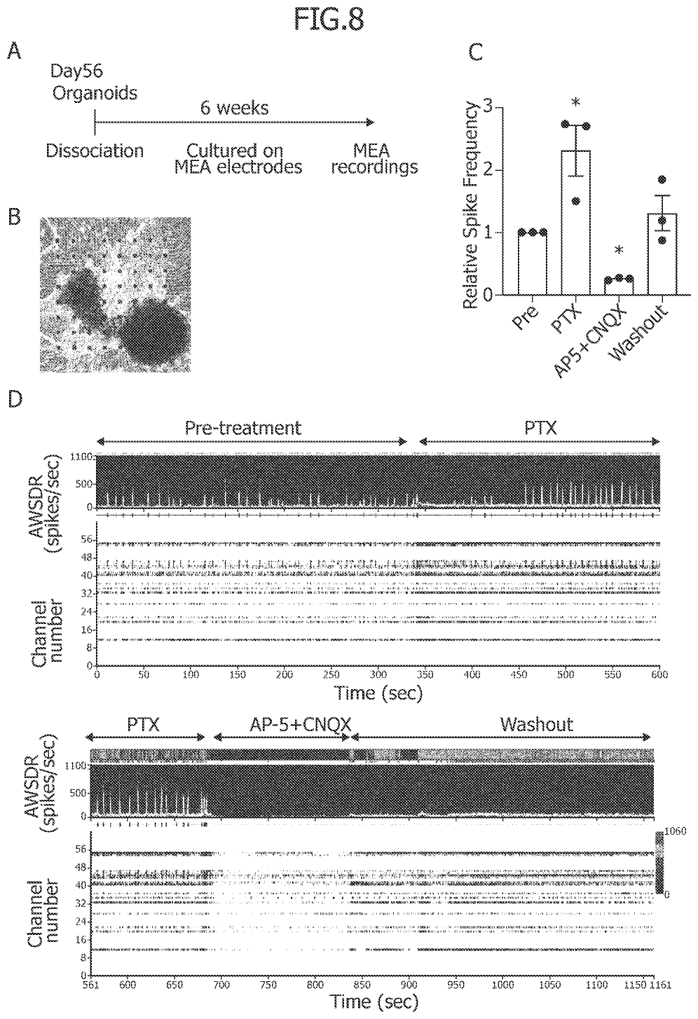

Brain organoids on Day 56 in which the neural functions of the neurons contained in the inverted brain organoids were analyzed using the MEA system were enzymatically treated, and the cell clusters (clumps) obtained were placed on MEA chips coated with polyethyleneimine and Laminin-511 (MED-R515A, Alpha MED Scientific Inc.) and cultured for 6 weeks (FIG. 8A). FIG. 8B shows a phase-contrast microscope image of the cell clusters cultured for 6 weeks. It can be seen that many neurites were elongated. The extracellular membrane potential of the culture system on the MEA chips was measured using a 64-channel MEA system (MED64-Basic, Alpha MED Scientific Inc.) to analyze the spontaneous firing frequency (Pre in FIGS. 8C and 8D). As shown in FIG. 8D, it was clarified that spontaneous firing was observed in each electrode, and synchronized burst firing occurred.

Therefore, it was shown that synaptic connections were formed between neurons in the culture system on the MEA chips, and that each neuron formed a network (nerve network) through the synaptic connections.

Thereafter, in order to check whether or not the synaptic connections were functional, the responsiveness to synapse agonists was analyzed. Specifically, in the culture system on the MEA chips, a GABA receptor antagonist (PTX) was added to the medium, glutamic acid receptor antagonists (APS and CNQX) were subsequently added thereto, and the medium was thereafter replaced to remove the antagonists. FIG. 8D shows the time-dependent changes in the extracellular membrane potential measured in this series of treatment processes, and FIG. 8C shows the relative values of the spike frequency in each process. It can be seen that the spike frequency remarkably increased after PTX treatment, and the spike frequency dramatically decreased after the treatment with APS and CNQX but was recovered by the removal of drugs. Therefore, it was shown that both synapses inhibited by PTX (that is, GABAergic inhibitory synapses) and synapses inhibited by APS and CNQX (that is, glutamatergic excitatory synapses) were formed in the culture system on the MEA chips.

These results showed that the neurons constituting the inverted brain organoid could form functional synapses and further could form a nerve network. Such data suggested that the inverted brain organoid was functionally mature.

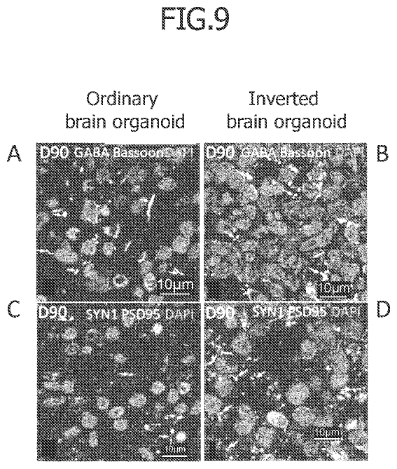

Then, the synapses in the brain organoid were analyzed, FIGS. 9A and 9B show the results of double immunostaining of the sections of the brain organoids obtained in Comparative Example 1 and Example 3, with GABA and inhibitory post-synaptic markers (Bassoon). Many GABA/Bassoon-double positive (yellow) dots were observed in both the conventional (A) and inverted (B) brain organoids (arrows in FIGS. 9A and 913). Furthermore, FIGS. 9C and 9D show the results of double immunostaining with an excitatory presynaptic marker (SYN1) and a post-synaptic marker (PSD-95). Many SYN1;PSD-95-double positive (yellow) dots were observed in both the conventional (C) and inverted (D) brain organoids (arrows in FIGS. 9C and 9D). Therefore, it was confirmed that synaptic connections were formed also in the inverted brain organoid, and it was strongly suggested that a nerve network was formed.

### (5) Analysis of Neural Stem Cells

In order to further understand the difference between the conventional and inverted brain organoids, analysis was performed with a focus on neural stem cells. As a result of various analyses using 3D-immunohistochemical staining, it was found that there was a significant difference in the proportion of cells having primary cilia, the length of the primary cilia, and the direction in which the primary cilia grow 3D-immunohistochemical Staining The brain organoid block was divided into two and subjected to membrane permeabilization (0.5% PBST, room temperature, 2 hours) and blocking treatment (0.1% PBST containing 10% normal goat serum, room temperature, 2 hour). Using an anti-ARL13B antibody (primary cilium marker, 17711-1-AP, Proteintech Group, Inc., dilution ratio:=1:1000) or an anti-SOX2 antibody (MAB2018, R&D Systems, Inc., dilution ratio 1:1000), primary antibody treatment (4° C., for 7 days) was performed, under stirring with a Rotator. Furthermore, using AlexaFlour 488 and 594 conjugates (Invitrogen, dilution ratio=1:1000), secondary antibody treatment (4° C., for 6 days) was performed under light-shielding conditions and stirring with a Rotator. The treated blocks were analyzed using a two-photon excitation microscope (Nikon AIR MP). 10148_1

### Results

A and Bin FIG. 10a show images obtained with the two-photon excitation microscope of the first cell layers of the conventional and inverted brain organoids, respectively. In A and B of FIG. 10a, green ellipsoidal signals represent individual neural stem cells, and yellow (SOX2.1Ar113b-double positive) signals observed inside the green signal represent primary cilia. It is understood that the inverted brain organoid (B in FIG. had a much higher number of yellow signals and shorter lengths thereof, as compared with the conventional type (A in FIG. 10a). From the results of quantifying the number and length of the yellow signals (G and H in FIG. 10b), it was clarified that the inverted brain organoid had a much higher proportion of neural stem cells having primary cilia (about 5% in the conventional type but about 50% in the inverted type) as compared with the conventional brain organoid with significantly shorter cilium lengths (about 5 μm in the conventional type but about 3 μm in the inverted type).

Furthermore, it was confirmed that there was a deviation in the directions in which the primary cilia grow in the neural stem cells of the conventional brain organoid. Neural progenitor cells generated from neural stem cells in the neuroepithelium migrate toward the basal side of the neuroepithelium. Furthermore, neurons generated from neural progenitor cells migrate toward the outside of the neuroepithelium (ventricular zone). Therefore, the “direction of neural differentiation (that is, the direction in which the cells differentiated from neural stem cells migrate)” can be approximated to the “normal vector at any point on the apical surface of the first cell layer”.

C to F in FIG. 10a, show images obtained by triple immunohistochemical staining of the conventional and inverted brain organoids with SOX2 (green),Ar113b (gray), and Pent (yellow: marker for the base of the primary cilia), respectively. Enlarged images of the regions surrounded by squares in C and D of FIG. 10a are F and F of FIG. 10a, respectively. The arrows (Directions of differentiation & migration) shown next to E and F of FIG. 10a represent the “directions of neural differentiation”. Pent/′SOX2-double positive (yellow green close to yellow) dots represent the bases of the primary cilia, and red signals extending from the dots represent the primary cilia.

Angles formed by the direction of neural differentiation and the signals of the primary cilia, starting from the bases of the primary cilia, are defined as “the directions in which the primary cilia grow” in SOX2-positive cells (I in FIG. 10c), and J in FIG. 1.0c shows the analysis results of such angles. From J in FIG. 10c, it is understood that the directions in which the primary cilia grow are often 0 degrees or 180 degrees, in the neural stem cells of the conventional brain organoid. In contrast, it was clarified that the directions in which the primary cilia grow were random, and the cilia were almost uniform over the entire 360° circumference without any significant deviation in the neural stem cells of the inverted brain organoid.

From FIG. 10d and FIG. 10e, the cells in the culture obtained by the vertical mixing method of Example 7 had the directions of the primary cilia changed as compared with those by orbital mixing. This indicates that hydrodynamic stimulation had changed the signals of the primary cilia. It can be understood that changes in the signals of the primary cilia lead to changes in Sonic Hedgehog Signals, as shown by the results of single-cell RNA sequencing, and these signal changes control the differentiation of stem cells into specific cell lineages.

It was suggested that, since the primary cilia were mechanosensors to receive various external stimuli, the difference described above might be involved in the expression of the inverted type. Furthermore, such data suggested that fluid dynamics within the bioreactor greatly influenced the inside-out or outside-in structure of iPSC-derived brain organoids via the directions of the primary cilia of neural progenitors.

### (6) Analysis of Organoid Shape

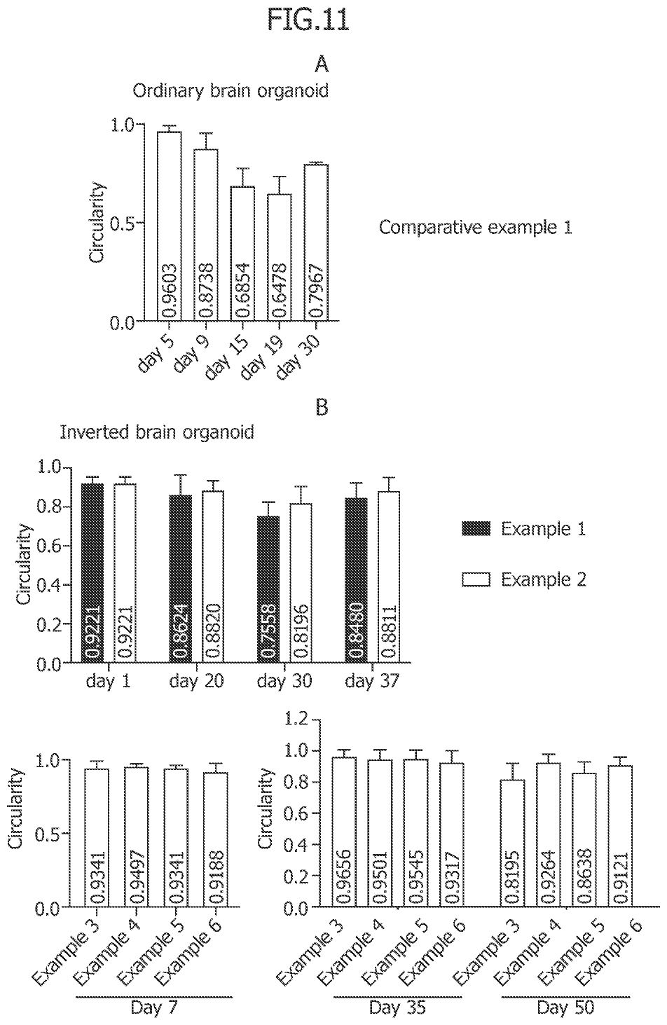

FIG. 11 shows the results of analyzing the shape (specifically, the sphericity) of the cultures produced by the methods of Comparative Example 1 and Examples 1 to 6. In the method according to the conventional art, it was confirmed that there was a period of time with a low sphericity (irregular shape) when brain organoids were generated from pluripotent stem cells (FIG. 11A). In contrast, it turned out that the sphericity of each culture was consistently high until brain organoids were generated from pluripotent stem cells, in the method according to the present invention (FIG. 11B). This difference is considered to reflect the difference in the physical force that the cells receive during the culture process, specifically, during unsteady stirring culture.

### (7) Fabrication of Pathology Models

Using the method of Example 3, brain organoids were produced from iPS cells of healthy subjects or familial Alzheimer's disease patients having the E693 deletion mutation in the APP gene. As a result of analyzing brain organoids two months later, brain organoids derived from healthy subjects and Alzheimer's disease patients were mostly inverted brain organoids (FIGS. 12A and 12B). Furthermore, as a result of immunostaining of organoid sections using an A13 oligomer-specific antibody, a large amount of A13 oligomers accumulation of was d in MAP2-positive cells (neurons) only in the patient-derived brain organoids (FIG. 12B). Therefore, it was shown that a pathology model of Alzheimer's disease having a feature of the inverted type could be produced by the method according to the present invention.

### (8) Hydrodynamic Simulation of Up-and-Down Unsteady Stirring Culture Computational Fluid Dynamics

Physical flow simulations in cell culture dishes/bottles were performed using a CFD software, and ANSYS Fluent 2019 R3 (ANSYS) was used tier calculations. A Realizable K-a model was used to verify the turbulence model. A VOF (Volume of Fluid) model was used for the gas-liquid interface of using a cell culture dish. Sliding wall conditions were applied to the gas-liquid surface using a bottle. The following formula was used to calculate the resistance.

$$D = \frac{1}{2} {ρ}_{i} {V}^{2} S {C}_{D} \qquad Expression 1$$

$${C}_{D} = \{ \begin{matrix}\frac{24}{Re} ( Re < 2 ) \\ \frac{10}{\sqrt{Re}} ( 2 < Re < 500 \\ 0.44 ( 500 < Re < {10}^{5} )\end{matrix}$$

$$Re = \frac{{ρ}_{f} u d}{{μ}_{f}}$$

### D: Drag (N)

pr: Fluid density (kg/m)
μf: Fluid viscosity (Pa-s)
V: Relative velocity of fluid and particle (m/s)
S: Cross-section of particle (m²)
C_{D}: Resistance coefficient (−)
Re: Reynolds number (−)
u: Particle movement speed (m/s)
d: Particle size (m)

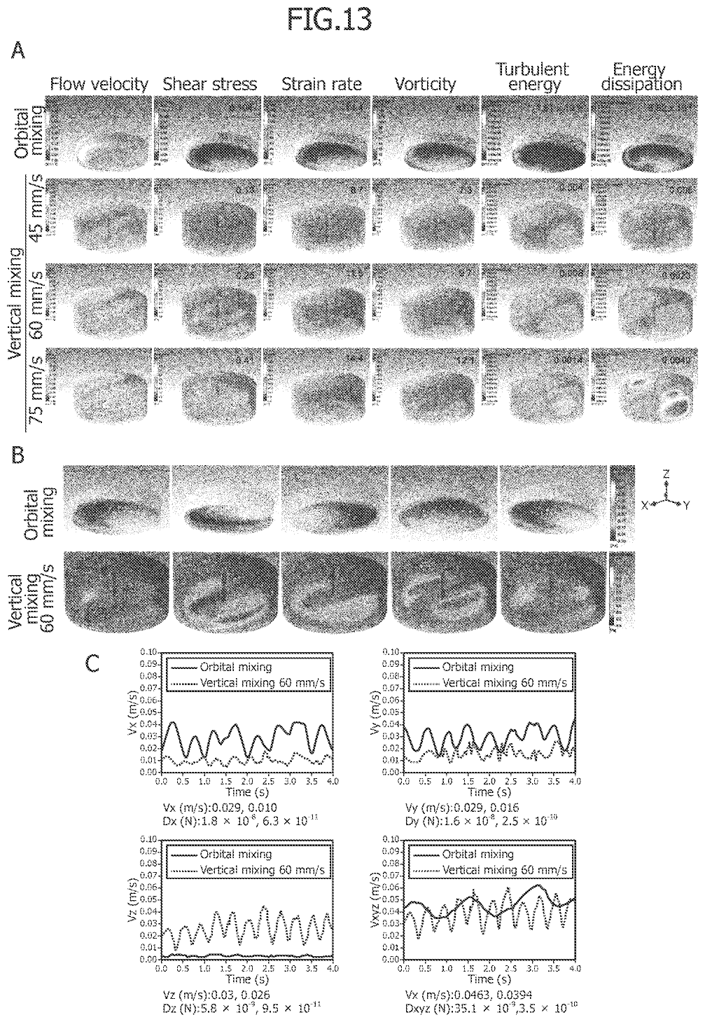

In order to understand the mechanism of inversion of the brain organoid structure, CFD analysis was conducted for both orbital mixing and vertical mixing. Using CFD analysis, the speed gradient in the culture dish, shear stress of each organoid, vorticity, and turbulence energy were calculated under three conditions (45 mm/s, 60 mm/s, and 75 mm/s) of orbital mixing and vertical mixing (FIG. 13A). The turbulence energy was greater during vertical mixing than during orbital mixing, suggesting that the difference in the turbulence energy might contribute to the formation of the inverted brain organoids. Furthermore, the flow rate of each organoid was calculated using solid-liquid mixed phase flow analysis. The dispersion in the orbital shaker was unequal, and the spheres were moving toward the center along the container wall (FIG. 13B, upper panel). In addition, the organoids during vertical mixing were uniformly dispersed throughout the entire culture tank (FIG. 13B, lower panel). Such flow characteristics and dispersion effects may have a favorable effect on homogenization of the organoids and may contribute to the generation of uniform structures by vertical mixing. Furthermore, a discrete phase model (DPM) showed differences in the flow rate between the organoids themselves during orbital mixing and vertical mixing and in resistance applied to the organoids. As a result of analyzing the three-dimensional flow rate, almost no migration of the organoids in the Z direction was observed during orbital mixing, whereas migration of the organoids not only in the X and Y directions, but also in the Z direction was observed during vertical mixing (FIG. 13C).

These results suggested that the formation of the inverted brain organoids obtained in this study might be due to the dispersing and leveling action of the organoids generated by vertical mixing and the low resistance applied to the migration of the organoids themselves. Furthermore, the turbulence energy of vertical mixing is greater than that of orbital mixing, suggesting that differentiation requires appropriate stimuli received by the cells from the fluid. CFD analysis suggested that the turbulence energy of the fluid might have a favorable effect on the formation of the inverted brain organoids. Furthermore, the organoids during vertical mixing are uniformly dispersed in the culture tank since the drag exerted on the organoids is low. That is, it was suggested that the brain organoids during vertical mixing were freely floating in a flow of a stress-free culture medium. Furthermore, iPSCs in the vertical mixing culture system may be endowed with early cues that are retained long term during differentiation into brain organoids.

### (9) Single-cell RNA Sequencing Data

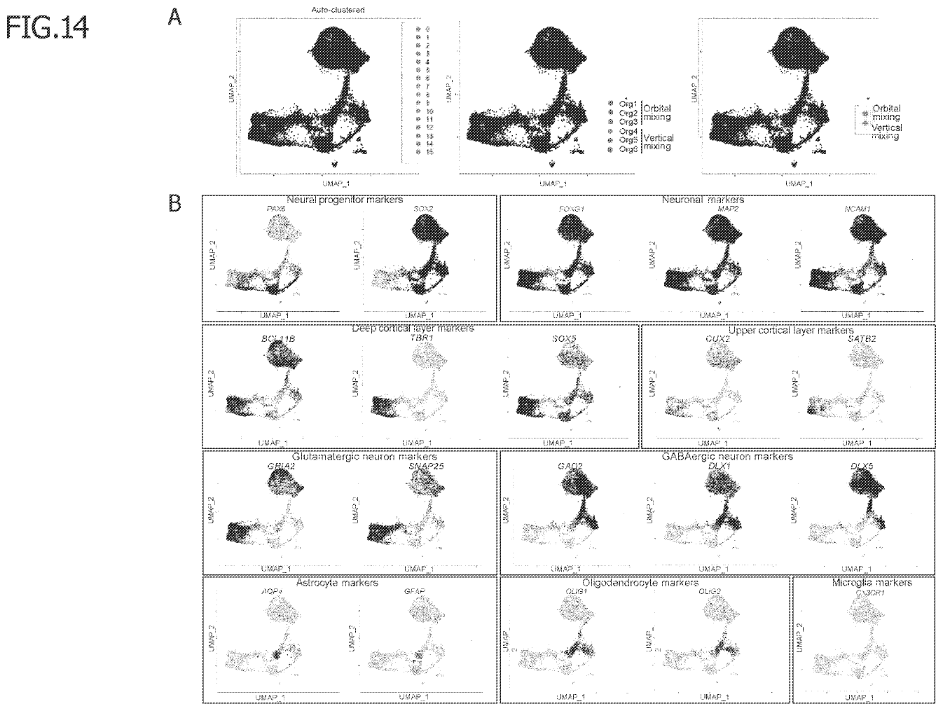

In order to analyze the cell types of the inverted brain organoids, the organoids of the Comparative Examples prepared by orbital mixing and the organoid of Example 7 prepared by vertical mixing (Day 90, n=3) were dissociated into single cells and subjected to single-cell RNA sequencing (scRNA-seq). A total of 12,000 cells were analyzed from 2,000 cells from each organoid. First, using the UMAP (Uniform Manifold Approximation and Projection) algorithm, the cells of both the organoids by orbital mixing and vertical mixing were aligned and co-clustered, and the expressions of the cells of the organoids were compared (FIG. 14A). UMAP is a conventional dimensionality reduction of the data matrix of the gene expression for each cell. In the process described above, K-means clustering on principal component analysis (PCA) space was used to define cell clusters, and the number of clusters was determined by the elbow method.

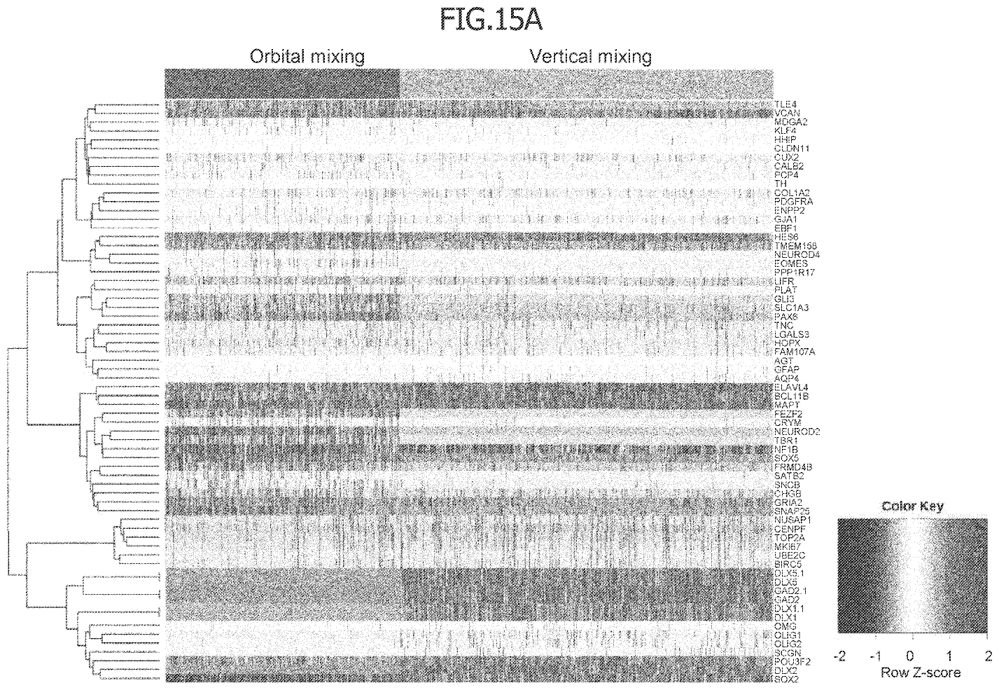

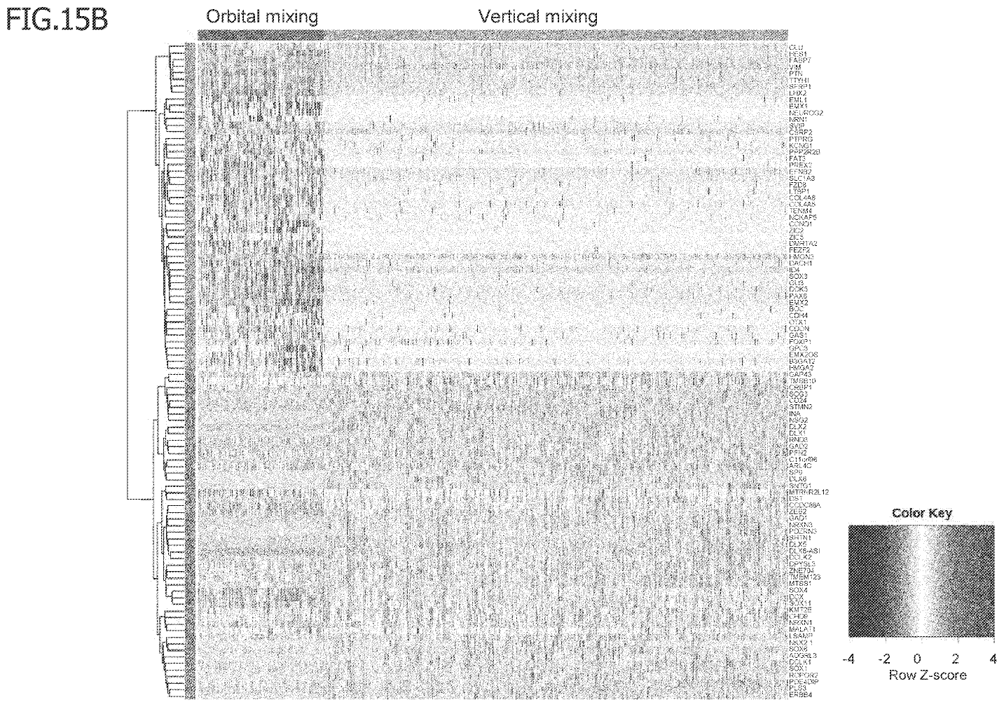

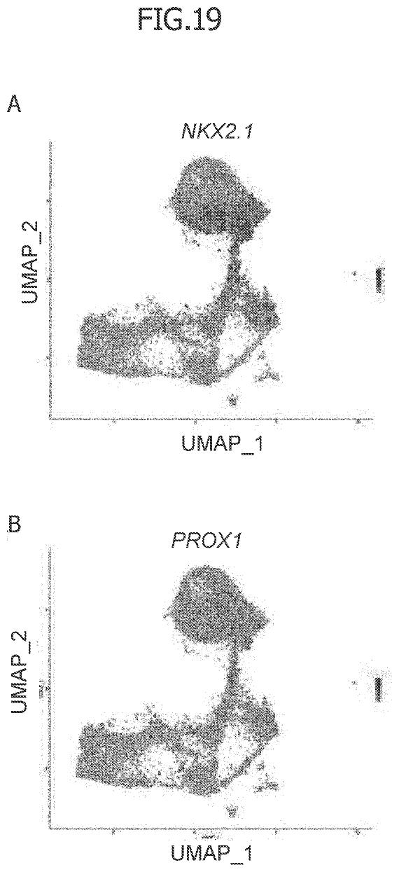

It can be understood from the detailed distribution by UMAP that neuronal marker-positive cells were abundant in both organoids by orbital mixing and vertical mixing (FIG. 14B). The inverted brain organoids also displayed markers for deep cerebral cortical neurons such as BCH 1B (also called CTIP2), TBR1, and SOX5 and markers for upper cerebral cortical neurons such s CUX2 and SATB2 (FIG. 14B). Furthermore, the expression of GABAergic marker genes (such as DLX1, DLX5, GAD, and NKX2.1) was confirmed in the inverted brain organoids (FIG. 14B and FIG. 19A), and it was suggested that a large number of GABAergic neurons are induced through Sonic Hedgehog Signaling. Furthermore, the heatmap showed differences in gene expression representing typical brain regions (FIG. 15a). These data suggested that under the stirring conditions of this example, the conventional brain organoid (Comparative Example 1) by orbital mixing had the identity of the dorsal forebrain, and the inverted brain organoid (the inverted type generated under the conditions of Example 7) by vertical mixing had the identity of the ventral forebrain. In this way, it turned out that there was a difference in the expression pattern of brain lesion-related genes between the brain organoids obtained in Examples and Comparative Examples, and the constituent cells of both organoids were quite different. More specifically, it was shown that the organoids obtained by the conventional method and the method of the present invention differ in composition of neurons and glial cells.

Therefore, it turned out that the proportion of GABAergic neurons was much higher, and the proportion of neurons in layer (TBR1-positive) and corticofugal neurons (SOX5-positive) remarkably decreased, in the unsteady stirring conditions (vertical mixing) of Examples, as compared with in horizontal rotary steady stirring (horizontal rotation on an orbital shaker) of Comparative Examples. These results suggested that different parts of the brain could be created simply by changing the stirring conditions without changing the medium composition.

Based on the finding of changes in the directions of the cilia shown in FIGS. 10a to 10d, a focus was given to cilia-associated signaling pathways in a population of SOX2-positive cells. The scRNA-seq data of SOX2-positive cells were analyzed in detail (FIG. 15b). In the SOX2-positive cells of the inverted brain organoid obtained in Example 7 (vertical mixing), it turned out that the expression of NKX2.1 increased, and Sonic Hedgehog Signaling was activated. Cilia-associated signaling was controlled by several factors related to the kinetics of Sonic Hedgehog Signaling. SOX2-positive cells showed alterations in gene expression such as GLI3, BOC, CDON, GAS123, and 24 for transmitting cilia-associated signaling (FIG. 15b). Furthermore, expressions of several cilia-associated genes such as CCDC88A and DCX were also altered (FIG. 15b). These results revealed that cilia-associated signaling was influenced by hydrodynamics in SOX2-positive cells. Accordingly, it was suggested that cilia-associated signaling in SOX2-positive cells may be altered by vertical mixing, and a characteristic structure called the “inverted type” may be formed.

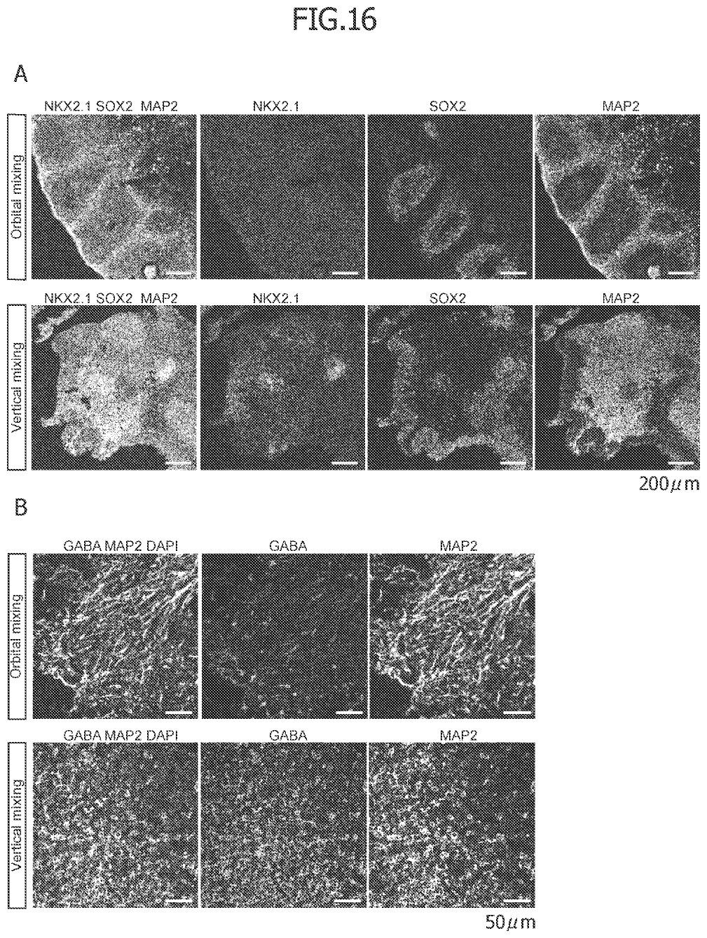

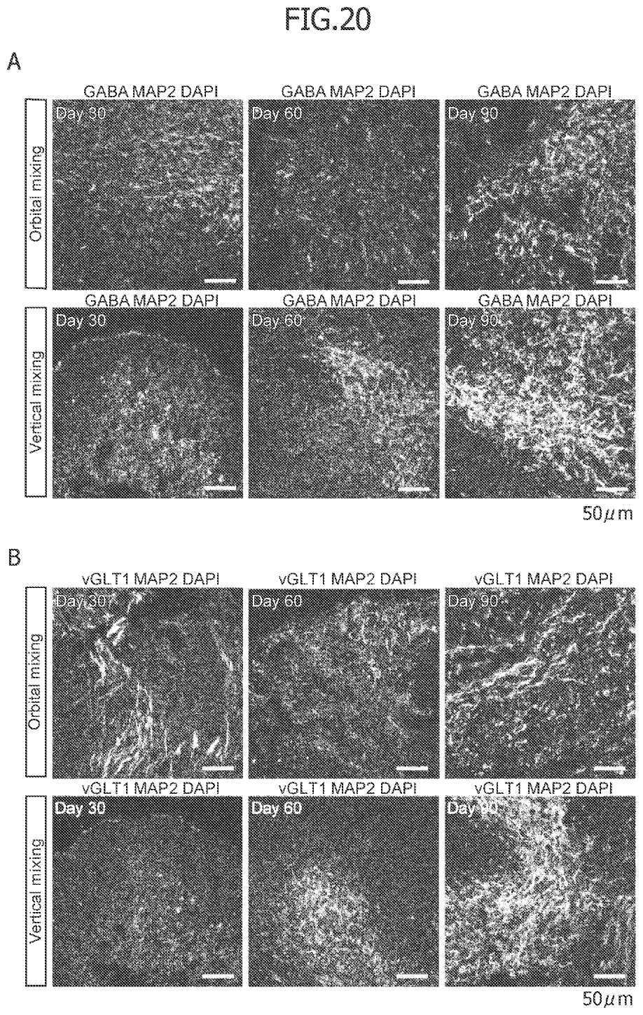

The results of immunostaining showed ventral neural progenitor cells on day 56 and GABAergic neurons on day 90 in both Comparative Example 1 (orbital mixing) and Example 7 (vertical mixing), but the number was overwhelmingly large in vertical mixing (FIGS. 16A and 16B), which agreed with the gene expression analyses (FIG. 14B, FIG. 15a, FIG. 15b, and FIG. 19). Furthermore, when the generation of excitatory neurons and GABAergic neurons in orbital mixing and vertical mixing along the time axis was evaluated, it turned out that vertical mixing may promote the differentiation of GABAergic neurons (FIG. 20). These data suggested that the inverted brain organoid was a characteristic brain organoid having a unique structure and a special cell composition. It also turned out from the scRNA-seq data that the expression of genes controlling cilia-associated signaling altered in SOX2-positive cells in association with Sonic Hedgehog Signaling, 10165_1

### (10) Analysis of Alzheimer's Disease Using Inverted Brain Organoid

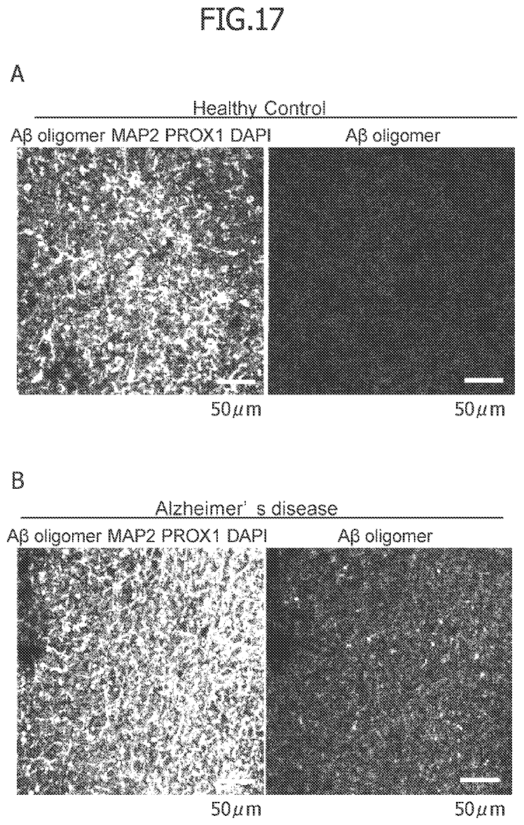

Several lines of evidence are emerging supporting the idea that alterations in GABAergic circuits contribute to the development of Alzheimer's disease (AD) by disrupting the function of the entire network. In order to use the inverted brain organoid for disease analysis, inverted brain organoids were prepared from iPSCs (APP E693A29) derived from healthy controls and familial AD patients lacking the APP protein E693. When cultured for two months by the method of Example 7 (vertical mixing), the brain organoids exhibited an inverted structure with the expression of the hippocampal marker PROX1, which agreed with the results of scRNA-seq analysis (FIG. 17 and FIG. 19B). That is, inverted brain organoids were obtained, and the generation of hippocampal neurons was confirmed. Accumulation of AP oligomers was observed in MAP2-positive neurons of AD brain organoids (FIG. 17B), and it was suggested that inverted brain organoids can serve as in vitro disease models.

### (11) Production of Organoid Without Using Matrigel

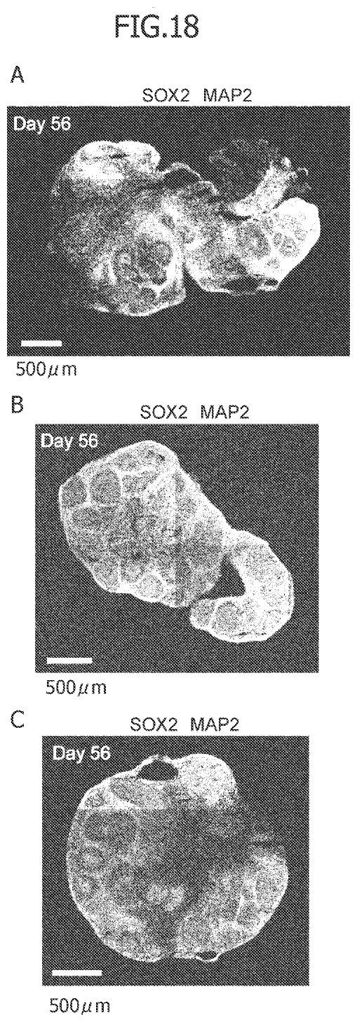

The effect of Matrigel on organoid structure was investigated without performing the step of embedding in Matrigel. An organoid was produced 1w the method of Comparative Example 1 (orbital mixing) without using Matrigel. The organoid generated was analyzed by immunostaining with SOX2 and MAP2 (FIGS. 18A, 18B, and 18C). The organoid obtained did not show an inverted pattern. The SOX2-positive region in the peripheral region, defined as within 100 μm from the edge of the brain organoid was 8.4±6.7% (n organoid, the mean±SD). It was shown that the generation of inverted structures was not promoted even without Matrigel. After vertical mixing, when orbital mixing was started on day 15, an inverted organoid was generated (data not shown in FIG. 18).

### (12) Generation of GABAergic Neurons and Glutamatergic Neurons

Generation of GABAergic neurons and glutamatergic neurons in orbital mixing and vertical mixing is shown (FIG. 20). GABAergic neurons stained with anti-GABA antibody were mainly observed in the organoid produced by vertical mixing (FIG. 20A), and glutamatergic neurons stained with anti-vGLT1 antibody were mainly observed in the organoid produced by orbital mixing (FIG. 20B). The scale bar represents 50

### (13) Culture Period by Vertical Mixing Indispensable for Preparation of Inverted Brain Organoid

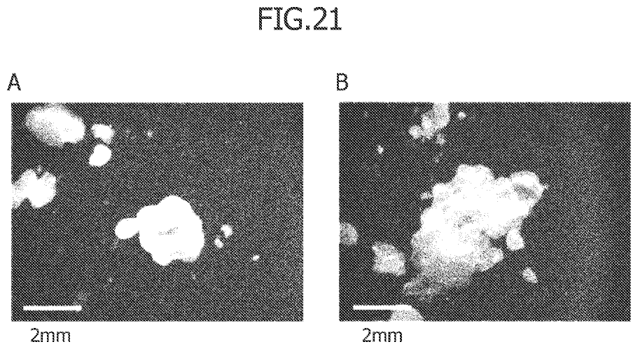

In order to directly examine the effect of vertical mixing on the formation of organoids, an experiment in which the stirring method was changed from orbital mixing to vertical mixing or from vertical mixing to orbital mixing on day 15 from the start of the differentiation was performed. In the experiment in which the stirring method was changed from orbital mixing to vertical mixing on day 15, all organoids were damaged, and culturing could not be performed for 40 days or more. This was considered to be due to the cells being vulnerable to mechanical stress. FIGS. 21A and B show images of damaged organoids.

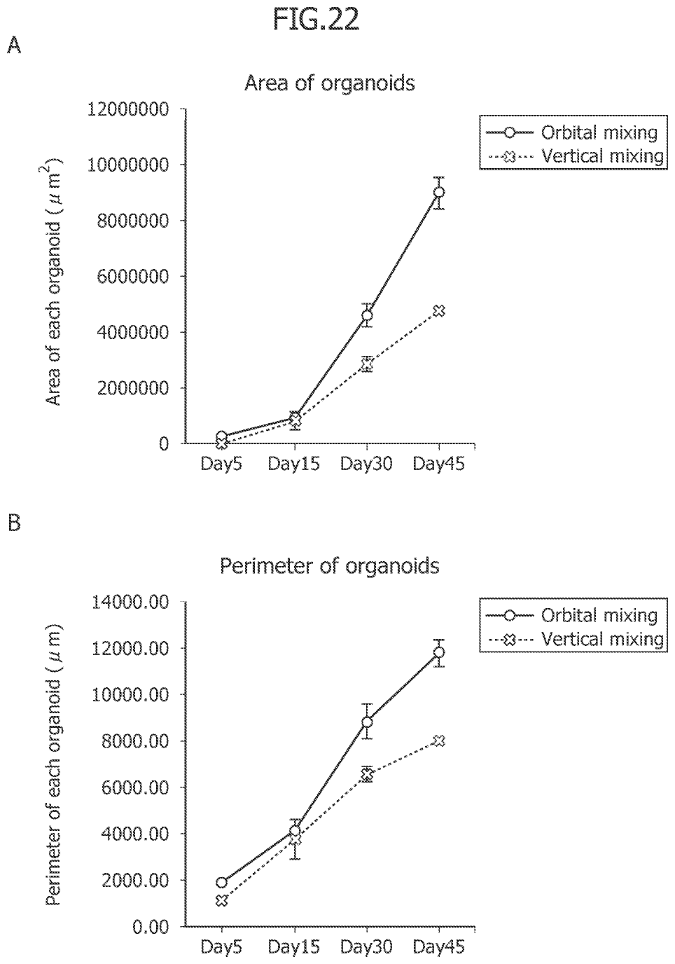

In order to examine the growth rate of the organoids, the area (A) and perimeter (B) of the organoids were quantified on the image (FIG. 22). Although the organoids generated by vertical mixing were smaller in size than the organoids generated by orbital mixing, they exhibited a similar growth rate to that of the organoid generated by orbital mixing. In each experiment, 30 organoids were evaluated. The data were shown as the mean±SD from three experiments.

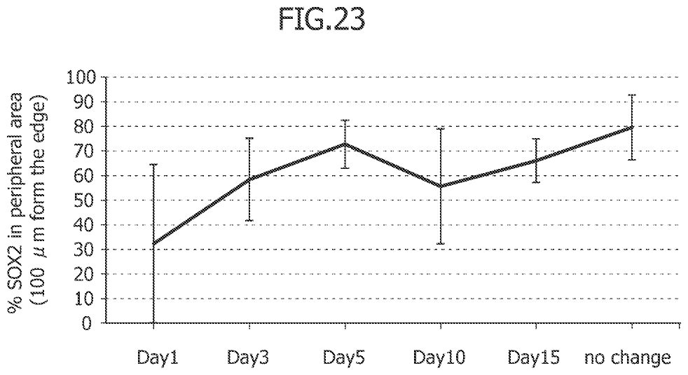

The cells were cultured under the stirring conditions (vertical mixing) of Example 7, the stirring conditions were switched to the stirring conditions (orbital mixing) of Comparative Example 1, after a certain period of time, and the cells were cultured until Day 56 to obtain cultured spherical bodies. Switching was performed on Day 1, Day 3, Day 5, Day 10, and Day 15, when the start of the differentiation was taken as Day 0. The cultured spherical bodies obtained were subjected to triple staining with SOX2, MAP2, and N-cadherin according to 2D-immunohistochemical staining described above, and the proportion of the SOX2-positive regions in the area up to 100 μm from the organoid surface was analyzed. FIG. 23 shows a graph of the results. Each point represents the mean±SD for three organoids.

From FIG. 23, even in the case in which the stirring conditions were switched on Day 1, the SOX2-positive regions in the superficial layer of the culture exceeded 30%, and a culture satisfying the definition of the “inverted brain organoid” of the present invention was obtained. When the stirring conditions were switched early, SOX2-positive regions were also observed remarkably in the second cell layer, but this decreased sharply when vertical mixing was performed until Day 15. Therefore, it was confirmed that unsteady stirring culture with up-and-down reciprocating motion was preferably performed until Day 10 (for the first 10 days), and unsteady stirring culture with up-and-down reciprocating motion was more preferably performed until Day 15 (for the first 15 days).

### (14) Production of Cultures Using HB9-EGFP Knock-in iPS Cell Lines (24-13)

A human iPS cell line in which the EGFP gene was inserted downstream of the promoter of the endogenous 1-1B9 gene (Mol Ther 2012 Feb; 20 (2): 424 to 31. doi: 10. 1038/mt. 2011. 266. Epub 2011 Dec 6.) was used to produce cultures. The cell line was provided by the author of the paper. Since this cell line expresses EGFP upon differentiation into spinal cord motor neurons, the cells that have differentiated into spinal cord motor neurons can be determined using the fluorescence of EGFP as an index. The culture conditions were the same as in Example 7, but the timing of replacing medium (1) with the medium (2) in FIG. 1D was changed from Day 6 to Day 7, and the timing of replacing the medium (3) with the medium (4) was changed from Day 25 to Day 24. The start of the differentiation was taken as Day 0, cultures were sampled on Day 7, Day 14, Day 34, and Day 58, cryosections were prepared, and immunostaining and the like were performed for analysis.

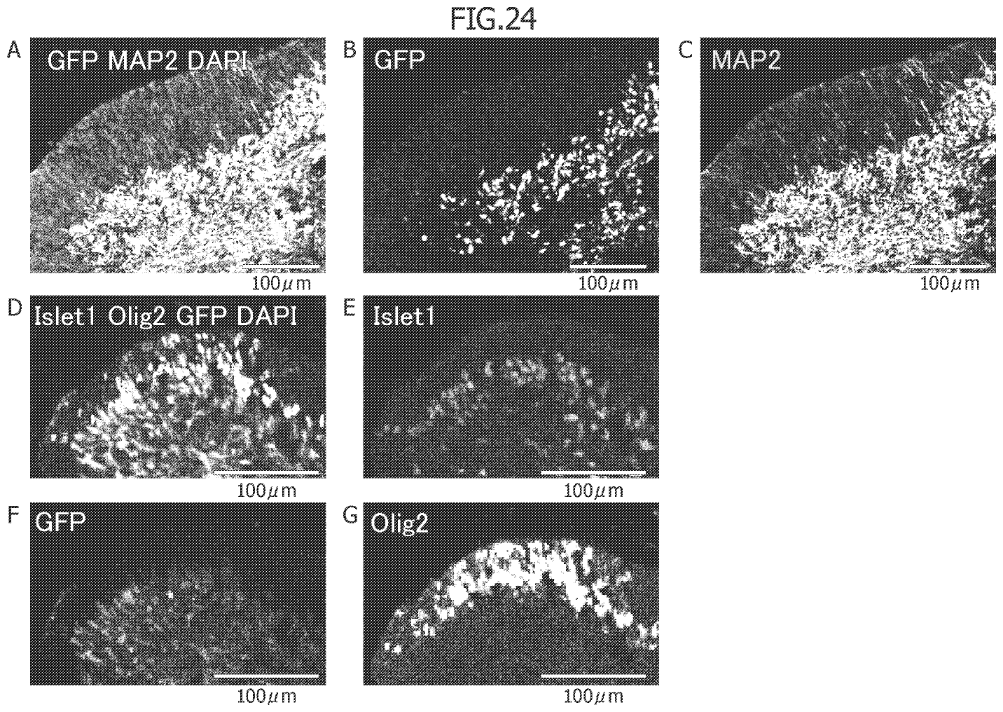

FIG. 24A is an image by merging GFP fluorescence (FIG. 24B, green), MAP2 immunostaining signal (FIG. 24C, white), and DAPI nuclear staining signal (blue) for organoids on Day 34. It can be seen that the second cell layer containing many MAP2-positive (white) neurons was present below the first cell layer heavily stained with DAPI (blue) (inside each organoid), and GFP-positive cells (that is, spinal cord motor neurons) were present inside the second cell layer comparatively densely. Furthermore, it was also confirmed from FIG. 24D that most part of the GFP-positive cells were ISL1-positive (red). According, it was shown that spinal cord motor neurons were generated on Day 34. Spinal cord motor neurons contained in cultures artificially produced from pluripotent stem cells can be used for cell transplantation therapy in subjects suffering from neurodegenerative diseases (such as ALS) in which spinal cord motor neurons are damaged and are very useful.

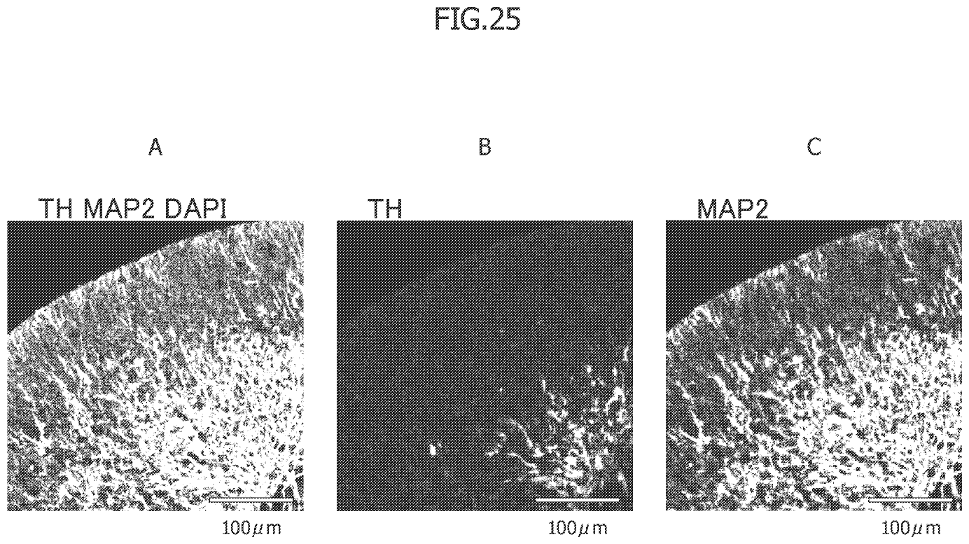

FIG. 25 is an immunostaining image showing the generation of dopaminergic neurons in organoids on Day 34 produced by vertical mixing. It can be seen that the second cell layer containing many MAP2-positive (green) neurons was present below the first cell layer heavily stained with DAPI (blue) (inside each organoid), and TH-positive cells (red), that is, dopaminergic neurons were present in a part of the second cell layer comparatively densely. Therefore, it turned out that dopaminergic neurons could be produced at an early stage of Day 34 by the method according to the present invention.

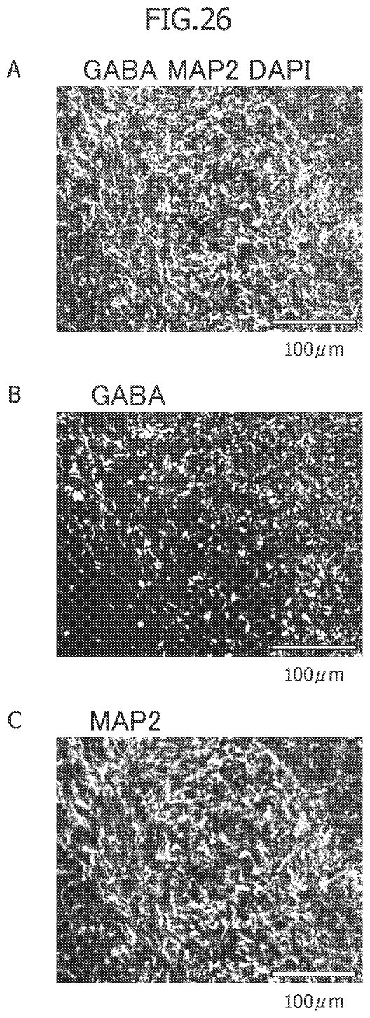

FIG. 26 includes images obtained by continuously using the medium containing retinoic acid at a concentration of 100 nM from the start of the differentiation and immunostaining of organoids produced by vertical mixing on Day 34. On Day 34, the generation of GABAergic neurons was confirmed. In cultures produced by a similar method without adding retinoic acid to the medium, the generation of GABAergic neurons was not confirmed on Day 34.

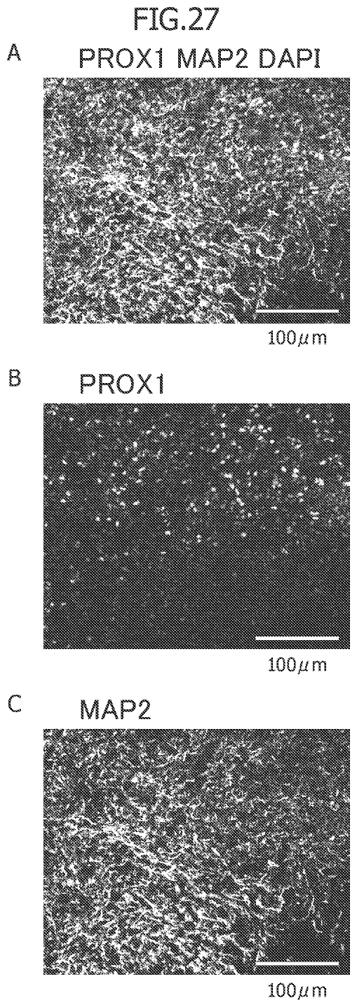

FIG. 27 includes images obtained by continuously using the medium containing retinoic acid at a concentration of 100 nM and immunostaining of organoids produced by vertical mixing on Day 34. On Day 34, a marker for hippocampal neurons, PROX1 was observed, and the generation of hippocampal neurons was confirmed.

It turned out from this that the time course of generation of GABAergic neurons and hippocampal neurons was remarkably accelerated by maintaining the medium containing retinoic acid from Day 0. The number and percentage of GABAergic neurons and hippocampal neurons generated in cultures on the same number of days increased as compared with cultures produced in a medium containing no retinoic acid.

Organoids produced by vertical mixing were subjected to immunostaining with a neural crest cell marker, SOX10, under the culture conditions of this example without containing retinoic acid. Although not shown in the figures, SOX10-positive cells were confirmed on Day 14, and the number had further increased by Day 34. In addition, SOX10-positive cells decreased by Day 58. The number of SOX10-positive cells increased from Day 10 to Day 34. Neural crest cells are also highly valuable cells for clinical applications such as cell transplantation therapy, and the fact that they can be artificially produced from pluripotent stem cells by the method of this example is of great significance.

### (15) Number of Days of Culture and Proportion of Cells Generated

The proportions of cells generated on each number of days of culture when cultured spherical bodies were produced by the methods of Example 7 and Example 8 was confirmed. In Example 8, the conditions were the same as in Example 7, except that the medium containing retinoic acid at a concentration of 100 nM was continuously used from the start of the differentiation. Sections of cultured spherical bodies from Day 7 to Day 90 obtained in each example were subjected to immunostaining using an antibody against each marker, and the proportion (%) of the antibody-positive region with respect to the total area of the sections is shown. When progenitor cell markers were present, they were used, and when progenitor cell markers were absent, only terminally differentiated cell markers were used. The number of cultured spherical bodies analyzed in each column was n>3. Table 6 shows the results. A blank in the table indicates that the analysis was not performed. Furthermore, the cultured spherical bodies obtained under the culture conditions of Example 8 were not subjected to immunostaining using antibodies against markers for glutamatergic neurons, dopaminergic neurons, progenitor cells of cerebellum cells, spinal cord motor neurons, neural crest cells, progenitor cells of photoreceptor cells, and progenitor cells of olfactory neurons.

TABLE 6

| Analysis | Cell type | GABAergic neuron | | Gluta- | Progenitor cell of | | Dopa- | Cerebellar | Spinal cord | Neural | Photo- | Olfactory |
|---|---|---|---|---|---|---|---|---|---|---|---|---|
| conditions | | (GABA) and its | | matergic | hippocampal neuron | | minergic | progenitor | motor | crest cell | receptor | neuron |
| | | progenitor cell | | neuron | | | neuron | cell | neuron | | progenitor | progenitor |
| | | (NKX2.1) | | | | | | | | | cell | cells |
| | Marker | NKX2.1, GABA | | vGlut1 | PROX1 | | TH | KIRREL2 | ISL1, HB9 | SOX10 | CHX10, | LHX2 |
| | | | | | | | | | | | CRX | |
| | Culture | Example 7 | Example 8 | Example 7 | Example | Example | Example | Example 7 | Example 7 | Example | Example 7 | Example 7 |
| | conditions | | | | 7 | 8 | 7 | | | 7 | | |
| Results | Day 7 | | | | | | | | | 5% | | |
| | Day 14 | | | | | | | | 5-20% | 30-50% | | |
| | Day 34 | <5% | 5-10% | 5-20% | <5% | 5-20% | 5-20% | 5-10% | 15-25% | 10-20% | 1-15% | 1-10% |
| | | (NKX2.1) | (NKX2.1) | | | | | | | | (CHX10) | |
| | Day 56 | 10-30% | | | 1-5% | | | 5-10% | | 1-10% | 1-10% | |
| | | (NKX2.1) | | | | | | | | | (CRX) | |
| | Day 90 | 50-80% | | | 5-10% | | | | | <1% | | |
| | | (GABA) | | | | | | | | | | |

From Table 6, it was confirmed that various types of brain neurons and peripheral neural cells were generated in various time courses within cultured spherical bodies produced by the method according to the present invention.

From the results of Examples above, it was shown that mechanical force by biomechanical engineering affects the differentiation ability of neural stem cells, and that brain organoids in which the positional relationship between the neural progenitor layer and the differentiated cell layer is inverted from the conventional brain organoids are generated as a result of applying the mechanical force. The inverted brain organoid according to the present invention can be applied to research on human brain development and disease.

Furthermore, it was confirmed that the inverted brain organoid according to the present invention is extremely useful as a supply source of neurons for transplantation.

### INDUSTRIAL APPLICABILITY

The cultured spherical body obtained by the present invention can be suitably used for research on differentiation and disease mechanisms, drug discovery screening, and the like, is useful as a supply source of neurons for transplantation, and can also be used as a pharmaceutical composition.

1. A cultured spherical body comprising:
a first cell layer containing neural stem cells and/or neural progenitor cells; and
a second cell layer containing brain neurons, wherein
the first cell layer is present in a superficial layer of the cultured spherical body.

2. The cultured spherical body according to claim 1, wherein the first cell layer has an apical-basal polarity, with the apical side of the first cell layer present on the surface layer side of the cultured spherical body.

3. The cultured spherical body according to claim 1, wherein the brain neurons contain at least one neuron selected from GABAergic neurons, dopaminergic neurons, and hippocampal neurons.

4. The cultured spherical body according to claim 1, wherein the second cell layer contains at least one neuron selected from spinal cord motor neurons and neural crest cells.

5. The cultured spherical body according to claim 1, wherein the proportion of the first cell layer in the superficial layer of the cultured spherical body is not less than 30%.

6. (canceled)

7. The cultured spherical body according to claim 1, wherein when the angle formed by the migration direction of each neural stem cell and the primary cilia is referred to as 0 in the plurality of neural stem cells contained in the cultured spherical body, the values of 0 randomly differ among the plurality of neural stem cells.

8. A method for producing a cultured spherical body from pluripotent stem cells, comprising the steps of:
(a) placing pluripotent stem cells in a culture tank with a stirring blade;
(b) unsteadily operating the stirring blade to perform unsteady stirring culture of the pluripotent stem cells in the presence of a pluripotent stem cell maintenance medium; and
(c) unsteadily operating the stirring blade to perform unsteady stirring culture of the pluripotent stem cells in the presence of a neuronal differentiation medium.

9. The method according to claim 8, wherein the unsteady operation step comprises an up-and-down reciprocating motion, a left-and-right reciprocating motion, a rotational motion with a variable speed, or a rotational reciprocating motion of the stirring blade, in the steps (b) and (c).

10. (canceled)

11. The method according to claim 8, wherein the unsteady operation step varies the operation of the stirring blade at an unsteady cycle in the range of 0.01 Hz to 100 Hz in the steps (b) and (c); or the unsteady operation step in the step (c) is performed for at least 10 days.

12. (canceled)

13. The method according to claim 8, wherein the neuronal differentiation medium contains retinoic acid or its derivative in the step (c).

14. A method for producing a cultured spherical body from pluripotent stem cells, comprising the steps of:
(i) placing pluripotent stem cells or undifferentiated cells derived from pluripotent stem cells in a culture tank with a stirring blade; and
(ii) unsteadily operating the stirring blade to perform unsteady stirring culture of the pluripotent stem cells or undifferentiated cells in the presence of a neuronal differentiation medium.

15. The method according to claim 14, wherein the unsteady operation step comprises an up-and-down reciprocating motion, a left-and-right reciprocating motion, a rotational motion with a variable speed, or a rotational reciprocating motion of the stirring blade, in the step (ii).

16. (canceled)

17. The method according to claim 14, wherein the unsteady operation step varies the operation of the stirring blade at an unsteady cycle in the range of 0.01 Hz to 100 Hz in the step (ii); or the unsteady operation step in the step (ii) is performed for at least 10 days.

18. (canceled)

19. The method according to claim 14, wherein the undifferentiated cells form cell clusters.

20. The method according to claim 14, wherein the neuronal differentiation medium contains retinoic acid or its derivative in the step (ii).

21. (canceled)

22. A pharmaceutical composition comprising the cultured spherical body according to claim 1, or a portion of the cultured spherical body.

23-25. (canceled)

26. A method for producing a spinal cord motor neuron, comprising the steps according to the method of claim 8.

27. A method for producing a spinal cord motor neuron, comprising the steps according to the method of claim 14.

28. A method for producing a GABAergic neuron or its progenitor cell, comprising the steps according to the method of claim 8.

29. A method for producing a GABAergic neuron or its progenitor cell, comprising the steps according to the method of claim 14.
